(19) United States
(12) Patent Application Publication
(10) Pub. No.: US 2026/0062738 A1
(43) Pub. Date: Mar. 5, 2026

(54) MICROFLUIDIC SYSTEM AND METHOD FOR DNA METHYLATION SAMPLE PREPARATION

(71) Applicant: University of Virginia Patent Foundation, Charlottesville, VA (US)

(72) Inventors: James P. Landers, Charlottesville, VA (US); Rachelle Ashley Turiello, Charlottesville, VA (US); Leah Michele Dignan, Charlottesville, VA (US)

(21) Appl. No.: 19/106,876

(22) PCT Filed: Aug. 25, 2023
(86) PCT No.: PCT/US2023/072938

§ 371 Date: Feb. 26, 2025

Related U.S. Application Data
(60) Provisional application No. 63/401,218, filed on Aug. 26, 2022.

(51) Int. Cl. C12Q 1/6844; B01L 3/00; B01L 7/00; C12Q 1/6806

(52) U.S. Cl. CPC C12Q 1/6844; B01L 3/502761; B01L 7/52; C12Q 1/6806; B01L 2200/0668; B01L 2200/16; B01L 2300/0832; B01L 2300/18; B01L 2400/0409

(57) ABSTRACT

Various aspects disclosed relate to a centrifugal microfluidic device to perform dynamic solid phase sodium bisulfate conversion. The device includes a reaction assembly. The reaction assembly includes a plurality of individual chambers, each including a bisulfate conversion chamber, an elution chamber, a magnetic manipulation chamber, a waste chamber, and a buffer chamber. The reaction assembly further includes at least one valve configured to selectively establish or prevent fluid communication along a channel between at least two respective individual chambers amongst the plurality of individual chambers. Additionally, the reaction assembly is configured to establish fluidic transport in response to rotation about an axis intersecting a central region of the reaction assembly and perpendicular to a plane on which the reaction assembly is disposed.

### CROSS-REFERENCE TO RELATED APPLICATION

This application claims the benefit of priority to U.S. Provisional Patent Application Ser. No. 63/401,218 entitled “MICROFLUIDIC SYSTEM AND METHOD FOR DNA METHYLATION SAMPLE PREPARATION,” filed Aug. 26, 2022, the disclosure of which is incorporated herein in its entirety by reference.

### BACKGROUND

Current forensic DNA analysis relies heavily on comparative approaches for human identification, in which unknown evidence samples are compared with known reference materials or database profiles. However, in many cases, references and known profiles from the Combined DNA Index System (CODIS) are not available.

### SUMMARY OF THE INVENTION

In some aspects, the techniques described herein relate to a centrifugal microfluidic device to perform dynamic solid phase sodium bisulfate conversion, the device including: a reaction assembly, including: a plurality of individual chambers including: a bisulfate conversion chamber; an elution chamber; a magnetic manipulation chamber; a waste chamber; and a buffer chamber; and at least one valve configured to selectively establish or prevent fluid communication along a channel between at least two respective individual chambers amongst the plurality of individual chambers; wherein the reaction assembly is configured to establish fluidic transport in response to rotation about an axis intersecting a central region of the reaction assembly and perpendicular to a plane on which the reaction assembly is disposed.

In some aspects, the techniques described herein relate to an in situ method for performing dynamic solid phase sodium bisulfate conversion, the method including: feeding a nucleic acid sample into a device, wherein device includes: a reaction assembly, including: a plurality of individual chambers including: a bisulfate conversion chamber; an elution chamber; a magnetic manipulation chamber; a waste chamber; and a buffer chamber; and at least one valve configured to selectively establish or prevent fluid communication along a channel between at least two respective individual chambers amongst the plurality of individual chambers; wherein the reaction assembly is configured to establish fluidic transport in response to rotation about an axis intersecting a central region of the reaction assembly and perpendicular to a plane on which the reaction assembly is disposed reacting the nucleic acid sample with sodium sulfate to form a partially sulphonated nucleic acid; spinning the device to move the partially sulphonated nucleic acid to the magnetic manipulation chamber to contact the partially sulphonated nucleic acid with a magnetic bead; deaminating and desulphonating the partially sulphonated nucleic acid in the magnetic manipulation chamber; contacting the deaminated and desulphonated nucleic acid with an elution buffer to form an eluted product; and spinning the device to move the eluted product to the elution chamber.

### BRIEF DESCRIPTION OF THE FIGURES

The drawings illustrate generally, by way of example, but not by way of limitation, various aspects of the present invention.

FIGS. 1A-1C are various views of microfluidic devices of the instant disclosure.

FIGS. 2A-2B are schematic representations of the in-tube “gold-standard” method described in the instant disclosure.

FIG. 3 is a block diagram illustrating an example of a machine upon which one or more aspects of the device can be implemented.

FIGS. 4A-4G are graphs showing assessments of DNA recovery and conversion efficiency of various aspects of Example 1.

FIGS. 5A-5D are views of a device of various aspects of Example 1.

FIGS. 6A-6D are various images and plots showing the results of a fluidic dye study using the device of Example 1.

FIGS. 7A-7F are various graphs showing DNA recovery and conversion efficiency of a device of Example 1.

FIGS. 8A-8B are graphs and plots showing the results of testing using the device of Example 2.

FIGS. 9A-9B are graphs showing PCR results for products generated using the device of Example 2.

FIGS. 10A-10B are graphs showing results for sulphonation/hydrolytic deamination using the device of Example 2.

FIG. 11 is a graph showing real-time PCR results for μCD sulphonation and hydrolytic deamination.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,”unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. A comma can be used as a delimiter or digit group separator to the left or right of a decimal mark; for example, “0.000,1” is equivalent to “0.0001.” All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt% to about 5 wt % of the composition is the material, or about 0 wt % to about 1 wt %, or about 5 wt % or less, or less than or equal to about 4.5 wt%, 4, 3.5, 3, 2.5, 2, 1.5, 1, 0.9, 0.8, 0.7, 0.6, 0.5, 0.4, 0.3, 0.2, 0.1, 0.01, or about 0.001 wt% or less, or about 0 wt%.

As used herein, a “DNA fragment” or “small DNA” or “short DNA” means a DNA that consists of no more than approximately 200 bp.

As used herein, the term “genome” refers to the genetic material (e.g., chromosomes) of an organism or a host cell.

As used herein, “sulfonated DNA” refers to the intermediate bisulfite reaction product that is a DNA comprising cytosines or uracils that have been sulfonated as a result of bisulfite treatment.

As used herein, a “small amount” of a DNA means less than about 100,000 molecules of that DNA or one or more DNAs having substantially the same functional sequence.

As used herein, the terms “hydrogen sulfite” and “bisulfite” are interchangeable.

As used herein, the terms “magnetic particles” and “magnetic beads” are used interchangeably and refer to particles or beads that respond to a magnetic field. Typically, magnetic particles comprise materials that have no magnetic field but that form a magnetic dipole when exposed to a magnetic field, e.g., materials capable of being magnetized in the presence of a magnetic field but that are not themselves magnetic in the absence of such a field. The term “magnetic” as used in this context includes materials that are paramagnetic or superparamagnetic materials. The term “magnetic”, as used herein, also encompasses temporarily magnetic materials, such as ferromagnetic or ferrimagnetic materials with low Curie temperatures, provided that such temporarily magnetic materials are paramagnetic in the temperature range at which silica magnetic particles containing such materials are used according to the present methods to isolate biological materials.

As used herein, the term “kit” refers to any delivery system for delivering materials. In the context of nucleic acid purification systems and reaction assays, such delivery systems include systems that allow for the storage, transport, or delivery of reagents and devices (e.g., inhibitor adsorbents, particles, denaturants, oligonucleotides, spin filters etc. in the appropriate containers) and/or supporting materials (e.g., buffers, written instructions for performing a procedure, etc.) from one location to another. For example, kits include one or more enclosures (e.g., boxes) containing the relevant reaction reagents and/or supporting materials. As used herein, the term “fragmented kit” refers to a delivery system comprising two or more separate containers that each contains a subportion of the total kit components. The containers may be delivered to the intended recipient together or separately. For example, a first container may contain materials for sample collection and a buffer, while a second container contains capture oligonucleotides and denaturant. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit”includes both fragmented and combined kits.

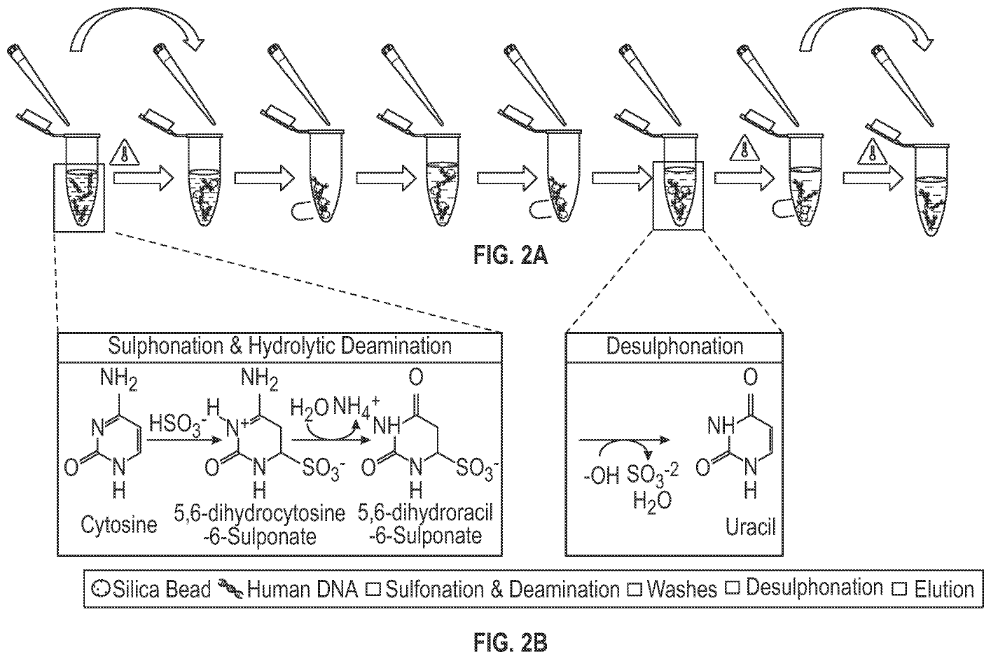

The approaches to forensic human identification (HID) are largely comparative in nature, predominantly relying upon the comparison of short tandem repeat (STR) profiles to known reference materials and/or database profiles. However, many STR profiles are generated from evidence materials that either do not have a reference material for comparison or do not produce a database hit. As an alternative to individualizing analysis for HID, researchers of forensic DNA have demonstrated that the human epigenome can provide a wealth of information related to sex typing, monozygotic twin differentiation, tissue individualization, body fluid identification, and human chronological age prediction. However, this type of analysis requires an additional sample preparation step, known as sodium bisulfite conversion (BSC), that is time-consuming, labor-intensive, prone to contamination, and characterized by extensive DNA loss and fragmentation. For forensic DNA laboratories, the addition of this epigenetic sample preparation workflow would increase time at the bench, provide more opportunities for analyst contamination and issues associated with interoperability, and result in a high degree of DNA loss or damage from samples that are often limited or fragmented prior to any analysis. In an effort to provide an alternative method for BSC that is more amenable to integration with the forensic DNA analysis workflow, the instant disclosure provides a rotationally-driven, microfluidic method for dynamic solid phase-BSC (dSP-BSC) that streamlines the sample preparation process in an automated format, capable of preparing up to four samples in parallel. The method was assessed for relative DNA recovery and conversion efficiency via real-time polymerase chain reaction (RT-PCR) and high resolution melting (HRM) and compared to a gold-standard BSC (shown schematically in FIG. 2) method and an enzymatic approach for cytosine deamination. Results indicate the microCD (μCD) method is capable of reducing incubation intervals by more than 36% and with comparable performance to a gold-standard approach, but with the potential for increased DNA recovery and conversion efficiency.

Current approaches to human identification (HID) of unknown persons remain largely comparative in nature, whereby short tandem repeat (STR) profiles from unknown evidence samples are compared with known reference materials/database profiles. Alternatively, unidentified human remains are morphologically categorized by visual interpretation by an anthropologist, as compared to discrete, published standards. Despite the statistical success of producing a match via STR analysis, many genetic profiles are generated from crime scenes, human remains, and sexual assault and evidence collection kits (SAECKs) that do not have a genetic reference material for comparison and do not produce a database hit. Further, the precision with which any trait is discerned via anthropological assessment for identification has been determined to be dependent upon the completeness of the remains and the anthropologist's prior experience. For these types of cases, the human epigenome has been suggested as a reservoir of information for sex typing, monozygotic twin individualization, body fluid identification, behavioral traits, and DNA phenotyping (FDP) by estimation of human chronological age. In particular, over the past 15 years more than 300 research studies and review articles have been published suggesting the utility of epigenetic methylation status at specified genetic loci for approximation of human age. Studies have demonstrated predictive success within 0.94 years from forensically-relevant body fluids including, but not limited to, blood, saliva, semen, and teeth. However, despite great research success and the forensic community's high regard for DNA-based testing, epigenetic age prediction has not been adapted into the forensic DNA analysis workflow or even used as a routine investigative technique by law enforcement personnel.

If adopted, the forensic epigenetic workflow would require an additional step during sample preparation, referred to as sodium bisulfite conversion (BSC), a method that has remained largely steadfast in its approach since its inception. Through a series of chemical modifications, the BSC process preferentially deaminates all unmethylated cytosines in the DNA transcript to yield uracil residues, leaving those cytosines containing a methyl group (e.g., 5-methylcytosines) intact and distinguishable for downstream analysis by methylation-specific real-time polymerase chain reaction (RT-PCR) or sequencing. Unfortunately, these techniques are characterized by extensive DNA loss; it has been shown that four of the most widely used BSC kits determined that the recovery from 200 ng of input DNA averaged as low as 33.2% and only as high as 55%. Further, these methods require time-consuming, labor-intensive workflows with a high propensity for contamination due to the multitude of open-tube pipetting steps. We conclude that adaptation of the epigenetic analysis workflow by the forensic community has stalled given the current requirement for large amounts of input DNA and the constraint that implementation of the associated laborious processes are not optimal for integration into the existing forensic DNA workflow.

Described herein is a microfluidic solution for forensic epigenetic sample preparation that leverages centrifugal force to enable rapid, efficient conversion of forensically-relevant DNA input masses in an automated microCD (μCD) format. Faster conversion rates are possible with the use of reduced reagent and sample volumes in chambers with an enhanced surface-area-to-volume ratio when compared with the conventional, in-tube BSC method; theories associated with miniaturization dictate that a system 1/10^{th} of the original reaction chamber size will result in 100-fold reduction in time, thus minimizing the need for long incubations. The use of centrifugal force as a mechanism for fluid movement is advantageous for forensics applications for three primary reasons.

First, this ensures that only a single mechanism is required for propulsion, eliminating the need for bulky external hardware (e.g., syringe pumps, electronics, tubing, etc.) that hinder portability and take up valuable bench space. Second, the mechanism permits automation in a fully closed system to mitigate contamination risk. Third, the forces controlling fluid movement through channels and into reaction chambers for precise chemistries are easily controlled by simply adjusting rotational speed, an aspect that may be coded for automation via a corresponding graphical user interface (GUI). With regard to automation specifically, the μCD approach is fully programmable via custom, external systems capable of heating, imparting rotational and magnetic forces at specified frequencies, and laser valving to open and close fluidic channels. For this application, the use of a silica dynamic solid phase (dSP) enables magnetically-actuated, bead-based conversion; together with careful consideration of fluidic architecture and valving strategy, this permits the completion of several sequential unit operations on-board. Conversion discs were designed to accommodate approximately 1/10^{th} of the fluid volumes required by conventional BSC methods and with a view of multiplexing in mind: that is, each μCD is capable of converting up to four samples per disc.

Microfluidic integration was assessed with standards and multiple downstream analytical processes, including RT-PCR, high resolution melting (HRM), and electrophoresis. Early phase goals of this project included testing the chemistry at the microfluidic scale, adjusting the parameters of the reaction steps associated with DNA loss, optimizing microfluidic architecture, and comparing μCD converted eluates with those originating from an in-tube, gold-standard method for conversion. For proof-of-concept, assay characterization was completed with primers targeting FHL2, a locus associated with age determination.

Results suggest the increased surface-area-to-volume ratio at the microscale enabled reduction of incubation intervals, thereby decreasing the total assay time, with some evidence of increased DNA recovery and comparable conversion efficiency to a so called “gold-standard method”.

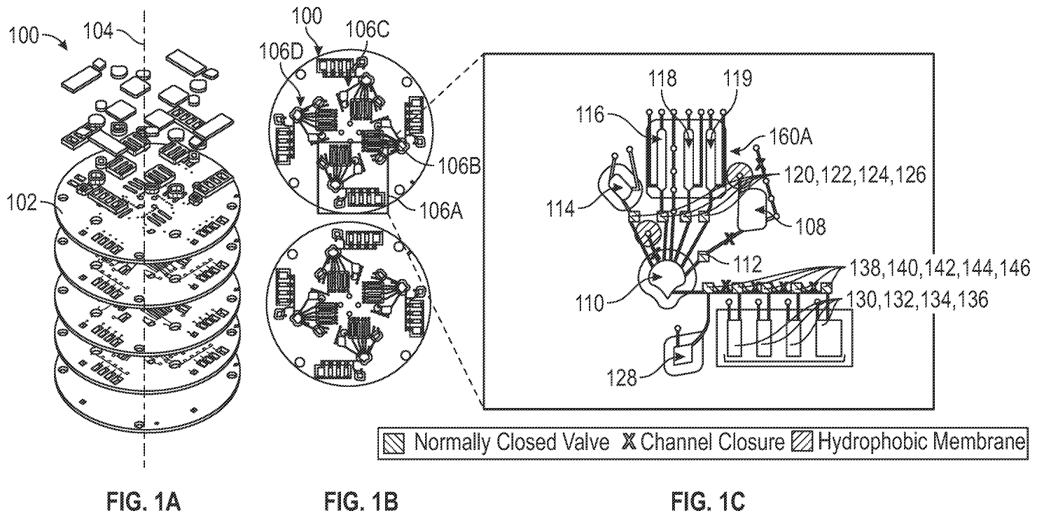

Various aspects of the presently disclosed subject matter relate to a centrifugal microfluidic device or kit to perform dynamic solid phase sodium bisulfate conversion. The device provides a useful alternative to the gold standard method. An example of centrifugal microfluidic device is depicted schematically herein at FIGS. 1A, 1B, and 1C. FIGS. 1A, 1B, and 1C depict many of the same components and are discussed concurrently. FIG. 1A is an exploded view of device 100. FIG. 1A shows 5-layer polymeric disc 102, rotational axis 104 and the individual components of reaction assemblies 106A, 106B, 106C, and 106D distributed about 5-layer polymeric disc 102.

FIG. 1C is a schematic view of reaction assembly 106A. Although reaction assembly 106A is discussed in detail, this discussion applies to reaction assemblies 106B, 106C, and 106D. As shown, reaction assembly 106A includes bisulfate conversion chamber 108. Bisulfate conversion chamber 108 is in fluid communication with magnetic manipulation chamber 110 with valve 112 located therebetween. Eluate buffer chamber 114, desulphonation buffer chamber 116, and wash buffer chambers 118 and 119 are in fluid communication with magnetic manipulation chamber 110, with valves 120, 122, 124, and 126, respectively, disposed therebetween. Magnetic manipulation chamber 110, is in further fluid communication with bisulfate conversion eluate chamber 128, and reagent waste chambers 130, 132, 134, and 136, with valves 138, 140, 142, 144, and 146, respectively disposed therebetween.

In operation, bisulphate conversion chamber 108 is loaded with a sulphonation reagent and a nucleic acid sample. The sulphonation reagent can be in a range of from about 5 μL to about 10 μL, about 10 μL to about 15 μL, less than, equal to, or greater than about 5 μL, 6, 7, 8, 9, 10, 11, 12, 13, 14, 15, 16, 17, 18, 19, or about 20 μL. An example of a suitable sulphonation reagent is available under the tradename Lightning Conversion Reagent™ available from Zymo Research of Irvine, California. The nucleic acid sample is DNA. The DNA can be a single stranded DNA (ssDNA) or double stranded DNA (dsDNA). The nucleic acid sample is present in a range of from about 0.5 μL to about 20 μL, about 1 μL to about 5 μL, less than, equal to, or greater than about 0.5 μL, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or about 20 μL. The volume of DNA is considered to be a small amount of DNA. The DNA can be a DNA fragment or short DNA in some examples.

Magnetic manipulation chamber 110 is loaded with a bead binding buffer and magnetic beads. The bead binding buffer can be in a range of from about 20 μL to about 80 μL, about 30 μL to about 50 μL, less than, equal to, or greater than about 20 μL, 25, 30, 35, 40, 45, 50, 55, 60, 65, 70, 75, or about 80 μL. The magnetic beads can be in a range of from about 2 μL to about 30 μL, about 5 μL to about 15 μL, less than, equal to, or greater than about 2 μL, 3, 4, 5, 6, 7, 8, 9, 10, 11, 12,13, 14, 15, 16, 17, 18, 19, 20, 21, 22, 23, 24, 25, 26, 27, 28, 29, or about 30 μL. The bead biding buffer can be about 6.5-7.5 M guanidine hydrochloride, about 3.6 M guanidine thiocyanate; 10 mM Tris HCl, pH 8.0; 40% 2-propanol. In some examples, the magnetic beads can include silica coating to bind DNA and the magnetic core provides an efficient way to concentrate and isolate the beads (and bound DNA) using a magnet. In some aspects, the silica-coated magnetic beads are MAGNESIL Paramagnetic Particles (Promega, Madison, Wis.; catalogue number AS1220 or AS640A, promega.com).

The disclosure is not limited to any particular type of magnetic bead. Examples of the technology described herein make use of any magnetic beads (e.g., paramagnetic beads) that have an affinity for nucleic acids. In some aspects, the magnetic beads have a magnetite (e.g., Fe₃O₄) core and a coating comprising silicon dioxide (SiO₂). The bead structure (e.g., size, porosity, shape) and composition of the solution in which a nucleic acid is bound to the bead can be altered to bind different types (e.g., DNA or RNA in single stranded, double stranded, or other forms or conformations; nucleic acids derived from a natural source, synthesized chemically, synthesized enzymatically (e.g., by PCR)) and sizes of nucleic acids (e.g., small oligomers, primers, genomic, plasmids, fragments (e.g., consisting of 200 or fewer bases) selectively. These characteristics of the beads affect the binding and elution of the nucleic acids to the beads. Also contemplated are magnetic beads coated with, e.g., organosilane; cellulose; hydroxysilane; and hydrophobic aliphatic ligands.

The disclosure is not limited to a particular size of magnetic bead. Accordingly, aspects of the technology use magnetic beads of a number of different sizes. Smaller beads provide more surface area (per weight unit basis) for adsorption, but smaller beads are limited in the amount of magnetic material that can be incorporated in the bead core relative to a larger bead. In some aspects, the particles are distributed over a range of sizes with a defined average or median size appropriate for the technology for which the beads are used. In some embodiments, the particles are of a relatively narrow monomodal particle size distribution.

In some aspects, the beads that find use in the present disclosure have pores that are accessible from the exterior of the particle. Such pores have a controlled size range that is sufficiently large to admit a nucleic acid, e.g., a DNA fragment, into the interior of the particle and to bind to the interior surface of the pores. The pores are designed to provide a large surface area that is capable of binding a nucleic acid. Moreover, in one aspect the disclosure is not limited to any particular method of nucleic acid (e.g., DNA) binding and/or isolation. Thus, some aspects of the technology relating to the bisulfate reaction are combined with other suitable methods of DNA isolation (e.g., precipitation, column chromatography (e.g., a spin column), etc.).

The beads (and bound material) are removed from a mixture using a magnetic field. In some embodiments, other forms of external force in addition to a magnetic field are used to isolate the biological target substance according to the present technology. For example, suitable additional forms of external force include, but are not limited to, gravity filtration, vacuum filtration, and centrifugation.

Examples of the technology apply an external magnetic field to remove the magnetic bead-DNA complex from the medium. Such a magnetic field can be suitably generated in the medium using any one of a number of different known means. For example, device 100 can position a magnet on the outer surface of magnetic manipulation chamber 110 holding a solution containing the beads, causing the particles to migrate through the solution and collect on the inner surface of the chamber 110. Commercial sources exist for magnets designed to be with device 100. See, e.g., MAGNESPHERE Technology Magnetic Separation Stand or the POLYATRACT Series 9600™ Multi-Magnet, both available from Promega Corporation; MAGNETIGHT Separation Stand (Novagen, Madison, Wis.); or Dynal Magnetic Particle Concentrator (Dynal, Oslo, Norway).

Each of wash buffer chambers 118 and 119 are loaded with a wash buffer. The wash buffer is present in an amount of about 20 μL to about 70 μL, about 30 μL to about 50 μL, less than, equal to, or greater than about 20 μL, 25, 30, 35, 40, 45, 50, 55, 60, 65, or about 70 μL. Examples of wash buffers include a mixture of about 80% ethanol and 10 mM Tris HCl at a pH of about 8.0. Desulphonation buffer chamber 116 is loaded with a desulphonation buffer in a range of about 20 μL to about 70 μL, about 30 μL to about 50 μL, less than, equal to, or greater than about 20 μL, 25, 30, 35, 40, 45, 50, 55, 60, 65, or about 70 μL. Examples of suitable desulphonation buffers include buffer available under the trade designation Zymo Research D5030-5 L-Desulphonation Buffer, from Zymo Research of Irvine, California. Eluate buffer chamber 114 is loaded with elution buffer in a range of about 15 L to about 50 μL, about 20 μL to about 30 μL, less than, equal to, or greater than about 15μL, 20, 25, 30, 35, 40, 45, or about 50 μL. Examples of elution buffers include a tris-acetate buffer (10 mM at pH 8) a TE buffer (10 mM trice-acetate at pH 8 and 1 mM EDTA).

Bisulphate conversion chamber 108 is positioned within reaction assembly 106A. The nucleic acid sample was exposed to two temperature intervals to complete the denaturation, sulphonation, and deamination steps (incubation). The first temperature interval ranged from about 90° C. to about 110 ° C., about 93° C. to about 110° C., less than, equal to, or greater than about 90° C., 91, 92, 93, 94, 95, 96, 97, 98, 99, 100, 101, 102, 103, 104, 105, 106, 107, 108, 109, or about 110 ° C. for about 0.3 minutes to about 5 minutes, about 0.5 minutes to about 1.5 minutes, less than, equal to, or greater than about 0.3 minutes, 0.5, 1, 1.5, 2, 2.5, 3, 3.5, 4, 4.5, or about 5 minutes. The second temperature interval ranges from about 40° C. to about 65° C., about 50° C. to about 60° C., less than, equal to, or greater than 40° C., 41, 42, 43, 44, 45, 46, 47, 48, 49, 50, 51, 52, 53, 54, 55, 56, 57, 58, 59, and 60° C. for a time ranging from about 35 minutes to about 55 minutes, about 40 minutes to about 50 minutes, less than, equal to, or greater than about 35 minutes, 36, 37, 38, 39, 40, 41, 42, 43, 44, 45, 46, 47, 48, 49, or about 50 minutes.

At the end of the aforementioned incubation, following incubation, valve 120 is opened and device 100 is spun to introduce the partially converted nucleic acid to magnetic manipulation chamber 110 for magnetic bead binding. Device 100 is spun at a rate of about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. The mixture is magnetically agitated for about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. The magnetic beads are subsequently pelleted a rate of about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes.

Following pelletization, valve 146 is opened, and device 100 is spun a rate of about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes to remove waste to chamber 136.

The magnetic beads are washed by introducing the wash buffer to magnetic manipulation chamber 110. This is accomplished by opening valve 126 to introduce wash buffer from wash buffer chamber 119 to magnetic manipulation chamber 110. To promote the flow of the wash buffer, device 100 is rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. Following magnetic mixing, for a time in a range of about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes, the magnetic beads are pelleted once again pelleting occurs at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes.

Valve 144 is then opened and device 100 is rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes to remove supernatant to waste chamber 134.

Desulphonation step begins by opening valve 112 to allow the desulphonation buffer to flow from bisulphate conversion buffer chamber 108 to magnetic manipulation chamber 110. After valve 112 is opened, device is rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. The resulting cocktail is magnetically mixed for a time ranging from about 0.3 minutes to about 2 minutes, about 0.5 minutes to about 1.5 minutes, less than, equal to, or greater than about 0.3 minutes, 0.4 0.5, 0.6, 0.7, 0.8, 0.9, 1.1, 1.2, 1.3, 1.4, 1.5, 1.6, 1.7, 1.8, 1.9, or about 2 minutes and held at about room temperature (e.g., 25° C.) for about 10 minutes to about 30 minutes, about 15 minutes to about 25 minutes, less than, equal to, or greater than about 10 minutes, 11, 12, 13, 14, 15, 16, 17, 18, 19, 20, 21, 22, 23, 24, 25, 26, 27, 28, 29, or about 30 minutes to complete conversion.

Waste from the desulphonation is removed following bead pelleting by opening of valve 140 and rotating device 100 at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.2 minutes to 3 minutes, about 0.3 minutes to about 1 minute, less than, equal to, or greater than about 0.2 minutes, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes.

The final wash occurs when valve 124 is opened and device 100 is rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.1 minutes to 3 minutes, about 0.2 minutes to about 1 minute, less than, equal to, or greater than about 0.1 minutes, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes to introduce the wash buffer to magnetic manipulation chamber 110. The mixture in magnetic manipulation chamber 110 is magnetically mixed for about 0.2 minutes to about 5 minutes, about 0.5 minutes to about 3 minutes, less than, equal to, or greater than about 0.2 minutes, 0.5, 1, 1.5, 2, 2.5, 3, 3.5, 4, 4.5, or about 5 minutes. The magnetic beads are pelleted by rotating device 100 rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.1 minutes to 3 minutes, about 0.2 minutes to about 1 minute, less than, equal to, or greater than about 0.1 minutes, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes.

Device 100 is rotated again at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.1 minutes to 3 minutes, about 0.2 minutes to about 1 minute, less than, equal to, or greater than about 0.1 minutes, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. To aide in evaporating the wash buffer, magnetic manipulation chamber 110 is placed between a dual-clamping Peltier system at a temperature in a range of from about 45° C. to about 65° C., about 50° C. to about 60° C., less than, equal to, or greater than about 45° C., 46, 47, 48, 49, 50, 51, 52, 53, 54, 55, 56, 57, 58, 59, 60, 61, 62, 63, 64, or about 65° C. for about 3 minutes to about 8 minutes, about 4 minutes to about 6 minutes, less than, equal to, or greater than about 3 minutes, 3.5, 4, 4.5, 5, 5.5, 6, 6.5, 7, 7.5, or about 8 minutes.

Elution of the nucleic acid is initiated when valve 120 is opened and device 100 is rotated at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.1 minutes to 3 minutes, about 0.2 minutes to about 1 minute, less than, equal to, or greater than about 0.1 minutes, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes to introduce elution buffer to magnetic manipulation chamber 110 and the beads.

Magnetic manipulation chamber is once again placed under the clamping system and heated to in a range of from about 45° C. to about 65° C., about 50° C. to about 60°C., less than, equal to, or greater than about 45° C., 46, 47, 48, 49, 50, 51, 52, 53, 54, 55, 56, 57, 58, 59, 60, 61, 62, 63, 64, or about 65° C. for about 3 minutes to about 8 minutes, about 4 minutes to about 6 minutes, less than, equal to, or greater than about 3 minutes, 3.5, 4, 4.5, 5, 5.5, 6, 6.5, 7, 7.5, or about 8 minutes. Once the nucleic acid has been released from the beads, they are once again pelleted by rotating device 100 at about 1000 rpm to about 3000 rpm, about 1500 rpm to about 2500 rpm, less than, equal to, or greater than about 1000 rpm, 1100, 1200, 1300, 1400, 1500, 1600, 1700, 1800, 1900, 2000, 2100, 2200, 2300, 2400, 2500, 2600, 2700, 2800, 2900, or about 3000 rpm for a time in a range of from about 0.1 minutes to 3 minutes, about 0.2 minutes to about 1 minute, less than, equal to, or greater than about 0.1 minutes, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 minutes. Valve 138 is opened to allow the nucleic acid to flow to bisulfate conversion eluate chamber 128 for pipette removal.

The development and characterization of a rotationally-driven microfluidic device for the dynamic solid phase sodium bisulfite conversion (dSP-BSC) of differentially-methylated human DNA is described. Preliminary characterization of the chemical workflow was accomplished in-tube and all iterative changes to the method were tested by comparing resultant eluates to those produced from the manufacturer recommended protocol to a corresponding ‘in-tube microfluidic’ protocol using reduced reagent volumes and incubation parameters. Likewise, BSC eluates produced following sample preparation via the ‘on-disc’ μCD approach were compared with those using the previously described in-tube approach. The selected target for early characterization is in the promoter region of FHL2, and is one associated with forensic human age prediction across multiple tissues. The primer sequences were previously vetted for PCR bias, function, and relevance to human age approximations.

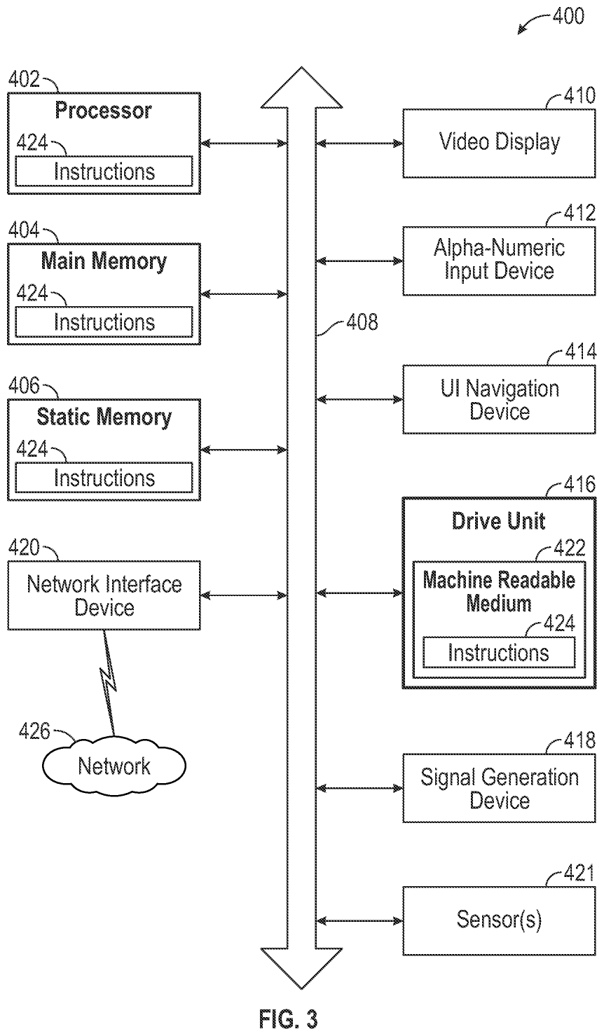

Referring to FIG. 3 an aspect of an embodiment of the present invention includes, but not limited thereto, a system, method, and computer readable medium that provides, in whole or in part, one or more of any combination of: a) rotationally-driven microfluidic system and method for DNA methylation sample preparation by dynamic solid phase sodium bisulfite conversion; b) optimization of a rationally-driven microfluidic system and method for forensic DNA methylation sample preparation (or other DNS applications besides forensic DNA); c) microfluidic system and method for DNA methylation sample preparation; d) Sodium Bisulfite Conversion (BSC) method is essential to enable incorporation of forensic DNA phenotyping (FDP), and ultimately reference-free externally visible characteristics (EVC) analysis, into forensic casework, for example (or other applications besides forensic); e) centrifugal microfluidic platform for dynamic Solid Phase sodium Bisulfite Conversion (dSP-BSC) that would enable rapid, efficient conversion of smaller, forensically-relevant DNA input masses in an automated, closed system (or other applications besides forensically-relevant DNA); f) fully integrated, automated, and enclosed system disclosed herein will minimize both variability and contamination risk through automation and integration to supplant the manual, open-tube steps required in traditional Sodium Bisulfite Conversion (BSC) methods; and/or g) rotationally-driven device for silica-facilitated sodium bisulfite conversion for downstream nucleic acid analysis, which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).) Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that can store, encoding, or carrying instructions for execution by the machine and that cause the machine to perform any one or more of the methodologies of the present disclosure or that can store, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of several transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that can store, encoding or carrying instructions for execution by the machine, and includes digital or analog communications signals or other intangible medium to facilitate communication of such software.

### EXAMPLES

Various aspects of the present invention can be better understood by reference to the following Examples which are offered by way of illustration. The present invention is not limited to the Examples given herein.

### Example 1

### Methods

Sample Materials. Preliminary testing of the in-tube and μCD dSP-BSC methods was accomplished with the Human Methylated & Non-Methylated DNA Set (Zymo Research, Irvine, CA, USA) at an initial concentration of 250 ng/μL to assess relative DNA recovery and BSC efficiency without potential variability resulting from DNA extraction. Universal Methylation Human DNA Standard (Zymo Research, Irvine, CA, USA) at a starting concentration of 20 ng/μL was used as a positive control for amplification and pyrosequencing; here, the positive control is fully methylated at all cytosine positions and bisulfite converted by the manufacturer. Negative controls were included during the BSC process, whereby human sample was substituted for nuclease free water and otherwise handled as if containing human genetic material. No template controls consisting of nuclease free water in place of the BSC eluate were included in all amplification and HRM detection modes.

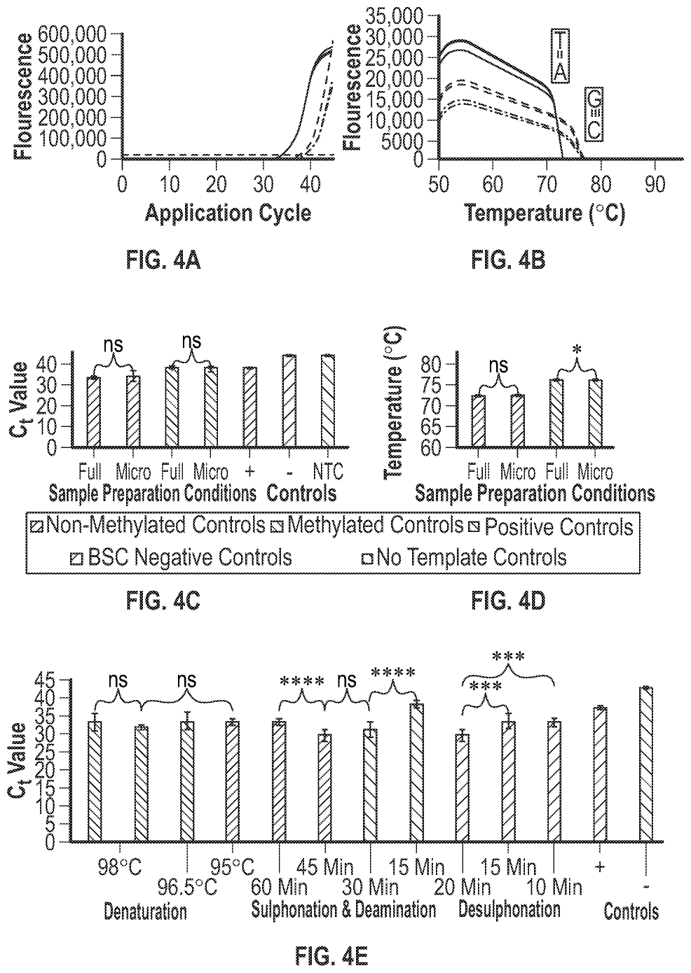

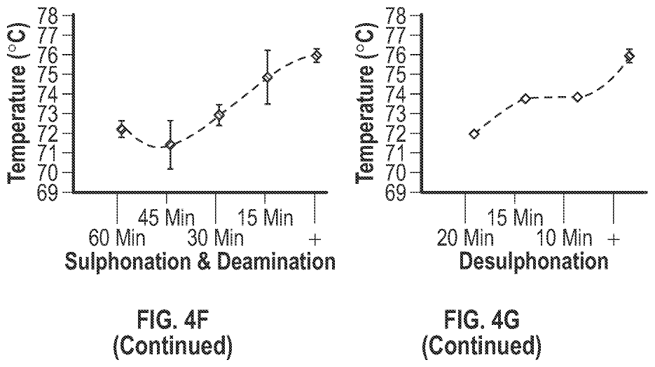

In-Tube dSP-BSC. For comparison with a gold-standard method, the dSP-BSC process was completed with the EZ-96 DNA Methylation-Lightning MagPrep (Zymo Research, Irvine, CA, USA) kit, according to the manufacturer recommended protocol, adapted for lower sample throughput (e.g., replacing the 96-well plate format with individual tubes and a magnet stand). For in-tube microfluidic reactions (FIGS. 4C-4G), volumes corresponding to the microdevice chamber capacities were used. Here, 2.5 μL DNA standard was added to 12.5 μL of Lightning Conversion Reagent (Zymo Research, Irvine, CA, USA) in a 0.2 mL tube; the 15 μL reaction mixture was heated on the Veriti thermal cycler (Thermo Fisher Scientific, Waltham, MA, USA) at 95° C. for 1 min and 54° C. 45 min for sulphonation and hydrolytic deamination. In separate 1.5 mL tubes, 40 μL of M-Binding Buffer and 10 μL MagBinding Beads were combined with the partially-converted DNA. Samples were mixed by vortexing, incubated at room temperature for 1 min, and placed on a magnetic stand before the supernatant was removed and discarded. The beads were then resuspended in 40 μL M-Wash Buffer, mixed by vortexing, and placed on the magnet stand for supernatant removal. Beads were then mixed with 40 μL L-Desulphonation Buffer, mixed by vortexing, and incubated at room temperature for 20 min Following Desulphonation, a second wash step was completed, as before, and the tubes were subsequently placed on a dry bath set to 55° C. for 1 min to remove residual M-Wash Buffer. Finally, the beads were resuspended in 25 μL of M-Elution Buffer, heated to 55° C. for 4 min and placed back on the magnetic stand. The BSC eluate was separated from the bead fraction by pipette and added to a 0.2 mL tube, which was then retained and stored at-20° C. until further analysis. For downstream detection via RT-PCR and HRM, a 5 μL volume of the BSC eluate was used, corresponding to a final PCR concentration of 5 ng/μL, except for the bead volume optimization study, wherein the final PCR concentration was 2 ng/μL. All in-tube BSC conversions were completed in technical replicates of 3 and PCR/HRM was also run in replicates of 3.

### Real-Time Polymerase Chain Reaction and High Resolution Melting

Amplification and detection of BSC eluates and corresponding controls was accomplished using the ZymoTaq™ DNA Polymerase (Zymo Research, Irvine, CA, USA) chemistry. Detection was made possible with the inclusion of an intercalating LAMP Fluorescent Dye (New England Biolabs, Ipswich, MA, USA). For conservation of reagents, half-reactions totaling 25 μL were used, including 12.5 μL 2× Reaction Buffer, 0.25 μL dNTP mix, 0.625 μL of 10 μM forward and reverse primers (Integrated DNA Technologies, Coralville, Iowa, USA), 0.2 μL ZymoTaq™ DNA Polymerase, 1.25 μL LAMP Fluorescent Dye, 4.55 L PCR-grade water, and 5 μL of BSC eluate, positive control, or nuclease-free water. All samples were run in triplicate on the QuantStudio 5 Real-Time PCR System with detection in the FAM channel (Thermo Fisher Scientific, Waltham, MA, USA). Thermal conditions included initial denaturation (95° C., 600 s), 45 cycles of denaturation (95° C., 30 s), annealing (50° C., 45 s), and extension (72° C., 60 s), and a final extension step (72° C., 420 s). For data analysis, eluates and controls were considered positive if they crossed the instrument-defined threshold, producing a Cₜ value. HRM was accomplished immediately following amplification on the QuantStudio 5 System and included thermal conditions whereby the reaction was denatured at 95° C. for 1 s, subsequently cooled to 50° C. and held for 20 s, before being incrementally heated to 95° C. at a rate of 0.1°C./s, with data acquisition occurring at each interval. The Tₘ of each sample was determined via the instrument's own algorithm. For visual clarity, some RT-PCR and HRM plots (FIGS. 4A-4B) were recreated in excel using raw fluorescence values extracted from the QuantStudio 5 system. To show the threshold line, baseline subtraction was calculated from cycles 3 through 15 and the threshold was plotted at three times the standard deviation of the mean baseline, as before.

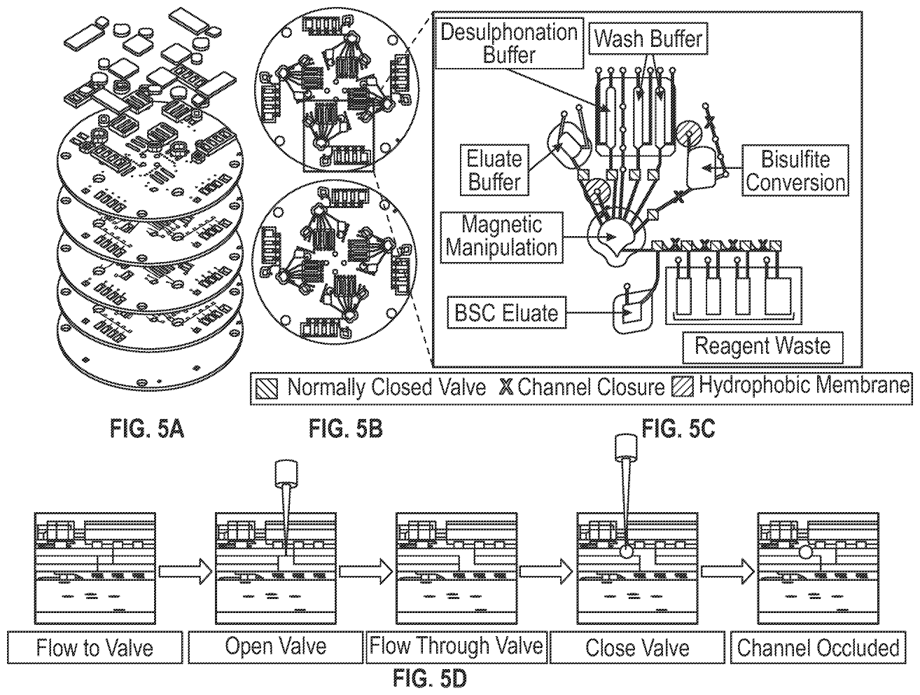

Operation of Mechatronic Systems. Spin systems to impart centrifugal force, enable laser-based valving to open and close fluidic channels, perform magnetic mixing, and on-disc heating were all designed in-house, as described previously. These systems are controlled by 8-core microcontrollers (Propeller P8X32A-M44; Propeller, Inc., Rockland, CA, USA) and corresponding, custom programs written in Spin and run from an external laptop. Rotational fluid propulsion and laser-based valving was accomplished with the Power, Time, and Z-Height Adjustable (PrTZAL) system. Here, valves were opened to permit flow into a new fluidic layer and into the corresponding chamber using laser power settings of 500 mW for an actuation time of 500 ms, and positioned 15 mm above the surface of the disc (FIG. 5D). Similarly, fluidic channels were closed by the same 638 nm laser diode to prevent backflow into the system using power, time, and z-height settings of 700 mW, 2500 ms, and 26 mm, respectively (FIG. 5D). A separate dynamic Solid-Phase Extraction (dSPE) platform was used to impart external magnetic control over the silica solid phase for efficient mixing of both DNA for capture and reagents for effective conversion. On-disc heating was accomplished with a dual-clamed Peltier system.

Microdevice Design and Fabrication. Iterative and final μCD prototyping was accomplished with AutoCAD software (Autodesk, Inc., Mill Valley, CA, USA). Designs were laser ablated into thermoplastic substrates and corresponding adhesives via a CO₂ laser (VLS 3.50, Universal Laser Systems, Scottsdale, AZ, USA). The core device contains five primary poly(ethylene terephthalate) (PeT) layers (Film Source, Inc., Maryland Heights, MO, USA); whereby primary fluidic layers (layers 2 and 4) are encapsulated with heat-sensitive adhesive (HSA) (EL-7970-39, Adhesives Research, Inc., Glen Rock, PA, USA). At the center of the μCD, a black PeT (bPeT) (Lumirror X30, Toray Industries, Inc., Chuo-ku, Tokyo, Japan) layer enables laser-based valving and provides a barrier between the two primary fluidic layers. Following alignment of the 5-layer device, layers were heat-bonded using a commercial-off-the-shelf laminator (UltraLam 250B, Akiles Products, Inc., Mira Loma, CA, USA). according to the “print, cut, laminate” method, described elsewhere. Multiple accessory pieces were added to the device via pressure-sensitive adhesive (PSA) transfer tape (MSX-7388, 3M, Saint Paul, MN, USA). Poly-(methyl methacrylate) (PMMA) (1.5 mm thickness, McMaster Carr, Elmhurst, IL, USA) capped with PeT was added to all chambers, not including the bisulfite conversion chamber, to increase chamber volume capacity. Polytetrafluoroethylene (PTFE) hydrophobic membranes (0.2 μm, Sterlitech, Auburn, WA, USA) were added to the vents of the bisulfite conversion and magnetic manipulation chambers to permit gas exchange during heated incubations on-board. Fluidic channels enabling flow from one chamber to another upon device rotation were designed to be approximately 100 μm deep and have widths between 400 and 500 μm.

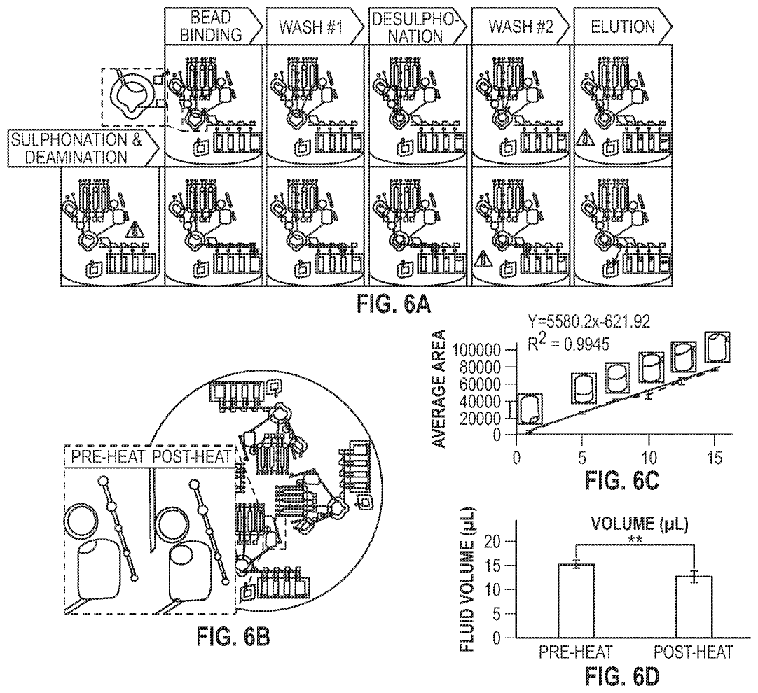

Fluidic Dye Studies and Corresponding Image Analysis. For early optimization of fluidic architecture, blue and yellow aqueous dye solutions were used to visually represent sample reagents (FIG. 6A). Following each workflow step (e.g., sulphonation and deamination), scanned images of the μCD were captured using an Epson Perfection V100 Photo desktop scanner (Seiko Epson Corporation, Suwa, Nagano Prefecture, Japan). Characterization of fluidic loss during the initial heating steps of the reaction was completed with 0.1 M Allura red dye solution (Sigma-Aldrich, St. Louis, MO, USA) diluted in 1× Tris-EDTA Buffer, pH 7.5 (Sigma-Aldrich, St. Louis, MO, USA) (FIGS. 6B-6D). Fiji Image J Freeware was used to evaluate fluid loss via ‘The Crop-Threshold-and-Go’ method of analysis. Briefly, cropped chamber images from digital scans were analyzed via the ImageJ color thresholding module to overlay a mask denoting the region of interest (ROI) from any background and providing a number of pixels associated with that mask. To build the calibration curve (FIG. 6D) and measure fluid loss pre-and post-heating (FIG. 6D), a total of 5 technical replicates were measured for each parameter.

Microdevice Dynamic Solid Phase Sodium Bisulfite Conversion. The complete μCD dSP-BSC process can be followed in the dye study detailed in FIG. 6A, which details the positions of all chambers and valves. The reaction begins with reagent and sample loading, wherein C1 is loaded with 13 μL Lightning Conversion Reagent and 2 μL of DNA sample. The neighboring C2 is loaded with a mixture of 40 μL Bead Binding Buffer and 10 μL Magnetic Beads. Chambers 4 and 8 are loaded with 40 μL of Wash Buffer and C6 is loaded with 40 μL of Desulphonation Buffer, while C10 is loaded with 25 μL of Elution Buffer. V1 is closed and C1 is positioned within the dual-clamped heating system for the following temperature intervals: 95° C. for 1 min and 54° C. for 45 min to complete the denaturation, sulphonation, and deamination steps. Following incubation, V2 is opened and the disc is spun (2000 rpm, 30 s) to introduce the partially converted DNA to C2 for bead binding. V3 is closed and the mixture is magnetically agitated on the dSPE system for 1 min Beads are subsequently pelleted (2000 rpm, 30 s), V4 is opened, and the disc is spun (1500 rpm, 30 s) to remove waste to C3, and V5 is closed. Wash #1 begins with the opening of V6 and disc rotation (1500 rpm, 15 s) to introduce Wash Buffer to C2. Following magnetic mixing (1 min), beads are pelleted once again (2000 rpm, 30 s), V7 is opened, and the disc is spun (1500 rpm, 30 s) to remove supernatant to C5 before V8 is closed. To begin the desulphonation step, V9 is opened and the disc is spun (1500 rpm, 15 s) to introduce Desulphonation Buffer from C6 to C2. The cocktail is magnetically mixed (1 min) and held at room temperature for 20 min to complete conversion. Desulphonation waste is removed following bead pelleting (2000 rpm, 30 s), the opening of V10, a spin step (1500 rpm, 30 s), and the closing of V11. The final wash occurs when V12 is opened and the disc is spun (1500 rpm, 15 s), introducing Wash Buffer into C2. The mixture is magnetically mixed (1 min), beads are pelleted (2000 rpm, 30 s), V13 is opened, the disc is spun again (1500 rpm, 30 s), and V14 is closed off to the upstream architecture. C2 is then placed between the dual-clamping Peltier system at a temperature of 55° C. for 5 min for Wash Buffer evaporation, prior to DNA elution. Elution is initiated when V15 is opened and the disc is rotated (1500 rpm, 30 s) to introduce Elution Buffer to C2 and the beads. V16 is closed and C2 is once again place under the clamping system and heated to 55° C., except for only 4 min. Once the DNA has been released from the beads, they are once again pelleted (2000 rpm, 30 s), V17 is opened, and the disc is spun to move the eluate from C2 to C11 for pipette removal.

Degradation Study. Degradation associated with the on-disc sample preparation method was assessed with the Quantifiler Trio Quantification Kit (Thermo Fisher Scientific, Waltham, MA, USA) according to the manufacturer recommendations and using the QuantStudio 5 Real-Time PCR System. Degradation indices were calculated by the HID Real-Time PCR Analysis Software (Thermo Fisher Scientific, Waltham, MA, USA) and were based upon the Cₜ values of diluted standards for large and small autosomal targets from 50-0.005 ng/μL according to manufacturer recommendations. Non-Methylated DNA standards were bisulfite converted using the on-disc μCD approach at a final concentration of 25 ng/μL in technical replicates of 3 and exactly 1 μL of converted eluate was used from each conversion replicate for evaluation of resultant degradation, equating to 1.25 ng/μL in each Quantifiler Trio reaction.

Enzymatic Methyl-Seq (EM) Conversion. A total of 13 μL Human Non-Methylated control DNA (Zymo Research, Irvine, CA, USA) was added to 117 μL 10 mM Tris-EDTA Buffer (Sigma-Aldrich, St. Louis, MO, USA), pH 8.0, for DNA fragmentation at a final concentration of 25 ng/μL. Shearing was completed using an S2 Ultrasonicator (Covaris, Woburn, MA, USA) with the 6×16 mm AFA Fiber microTubes (Covaris, Woburn, MA, USA) and settings associated with mean fragment sizes of 1.5 kilobases (kb) for a downstream application in RT-PCR and HRM, per manufacturer recommendations. The requisite volumes of sheared DNA were mixed with 10 mM Tris-EDTA Buffer to a total volume of 28 μL to begin conversion and amplify converted product to a final DNA input amount of 100, 10, and 1 ng of total input DNA in technical replicates of 2. The NEBNext® Enzymatic Methyl-Seq Conversion Module (New England Biolabs, Ipswitch, MA, USA) was used for enzymatic conversion according to the manufacturer's protocol and with Hi-Di Formamide (Applied Biosystems, Waltham, MA, USA) for denaturation and NEBNExt® Sample Purification Beads (New England Biolabs, Ipswitch, MA, USA) for purification. Subsequent amplification and HRM of converted eluates was completed as described here previously for the FHL2 target in replicates of 3.

Statistics and Reproducibility. All statistical calculations related to significance testing were completed with GraphPad Prism Software (San Jose, CA, USA). Cₜ and Tₘ values were described as the mean±standard deviations for all technical BSC replicates and/or amplification replicates. All described t-tests are two-tailed, using unpaired comparison parameters, and with a significance (α) of 0.05 (e.g., 95% confidence interval). Any analysis of variance (ANOVA) used a one-way framework and with the same 95% confidence interval parameters.

Dynamic Solid Phase Sodium Bisulfite Conversion Workflow. The conventional, ‘gold-standard’ BSC workflow was developed according to the manufacturer's protocol but modified for in-tube sample preparation (FIG. 2A). During the initial incubation, samples are heated to facilitate complete denaturation and subsequent progression of unmethylated cytosines through two intermediate structures, including 5,6-dihydrocytosine-6-sulphate and 5,6-dihydrouracil-6-sulphonate via ammonium bisulfite; this phase is referred to collectively as sulphonation and hydrolytic deamination (FIG. 2B). Following a bead wash and immobilization step, desulphonation occurs, forming uracil residues via incubation in a sodium hydroxide solution. Elution of the chemically converted DNA from the dynamic solid phase is completed following another bead wash (FIG. 2B). Following the reaction, only unmethylated cytosines are converted to uracil; methylated cytosines remain intact, as the addition of a methyl group to the ring contributes to stabilization of the structure and a lack on conversion due steric hindrance and electrostatic repulsion.

Downstream Analysis Strategy by RT-PCR and HRM. To assess the analytical performance of the upstream sample preparation method, multiple techniques were used to measure relative DNA recovery and conversion efficiency. Relative DNA recovery was evaluated via RT-PCR, whereby resultant cycle threshold (Cₜ) values were compared. Because these values are representative of starting concentration, it follows that samples prepared with optimal BSC conditions for DNA preservation would produce more rapid amplification (e.g., lower Cₜ values). Here, the ZymoTaq™ DNA Polymerase chemistry was used, as it was specifically designed for the amplification of bisulfite-treated DNA; however, the protocol was modified for reagent conservation and to enable real-time detection by utilizing only half-reactions and adding an intercalating Syto 9 dye, respectively. For verification of this detection method, methylated and non-methylated DNA standards were bisulfite converted in triplicate along with BSC negative controls and using the manufacturer's adapted protocol described above. Resultant eluates were successfully amplified along with methylated positive controls (previously converted by the manufacturer) and no template controls; non-methylated standards produced Cₜ values of ˜33.9 (±0.60) and methylated standards and converted positive controls produced values of ˜38.99 (±0.54) and ˜38.33 (±0.13), respectively. Noticeably, there was no statistical difference detected between the methylated standard converted in-house and with the modified in-tube approach and the positive control previously modified by the manufacturer (unpaired t-test, α=0.05, p-value=0.7446). While this may not signify that the modified ‘gold-standard’ method performs comparably in terms of conversion, since this is a fully methylated standard and conversion is not taking place, this does indicate that the methods are comparable in recovery (e.g., degradation). In addition, all samples were amplified with an initial concentration of 5 ng/μL per reaction; however, there is a reproducible shift in Cₜ units between the non-methylated and methylated samples. This shift may be explained by PCR bias, whereby the GC content of the methylated template is higher than that of the non-methylated sample post-conversion, leading to a comparatively diminished amplification product.

Likewise, relative conversion efficiency was demonstrated with HRM analysis, whereby the Tₘ of non-methylated and methylated control samples post-conversion was determined, and the corresponding differences were associated with a shift in GC content. Assuming 99-100% conversion efficiency with the gold-standard method, as is alleged, would dictate that all unmethylated cytosines are converted to uracil and then to thymine following PCR, thus, these transcripts should consistently exhibit a much lower Tₘ than their methylated counterparts. However, if BSC conditions are such that conversion efficiency becomes diminished, the Tₘ of unmethylated amplicons will undoubtedly shift upward, approaching that of the methylated sequences with higher GC content. As a baseline, non-methylated and methylated amplicons melted at temperatures of ˜72.39° C. (±0.16° C.) and ˜76.51° C. (±0.11° C.), respectively, indicating that, post conversion, non-methylated standards will have a lowered GC content compared with that of methylated standards, due to the overall reduction in hydrogen bonds in the template. For additional confirmation of the HRM method, methylated positive controls, previously converted by the manufacturer, also showed reproducible melt temperatures at ˜76.08 (±0.23° C.). Moving forward, if a statistically significant difference is detected for those non-methylated transcripts that have been bisulfite converted, it may be assumed that conversion efficiency has be altered. However, it is important to note here that HRM is only a measure of relative conversion efficiency and cannot be used to calculate the precise percentage (0-100%) typically associated with this metric.

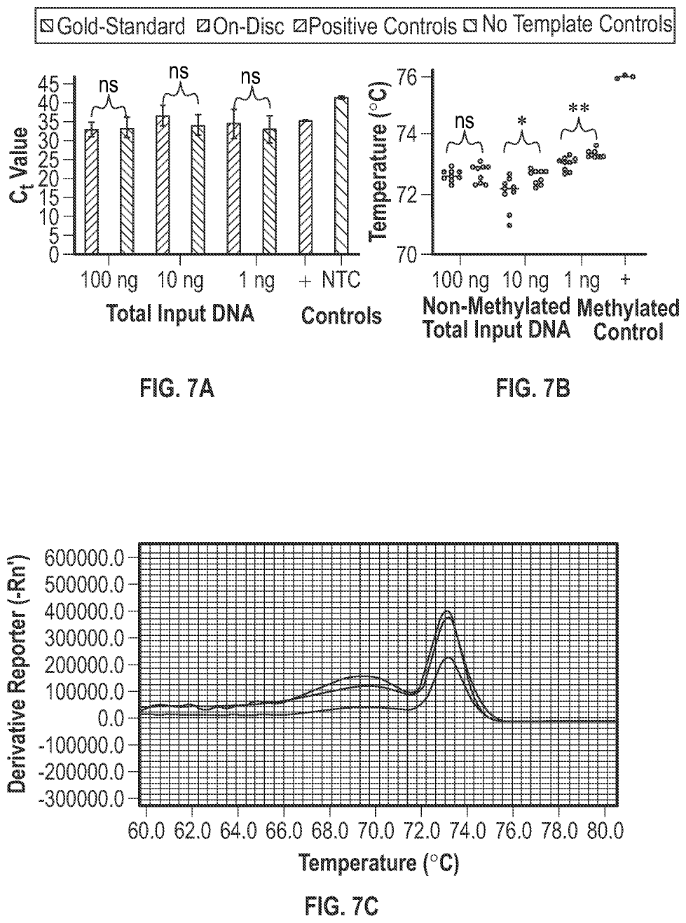

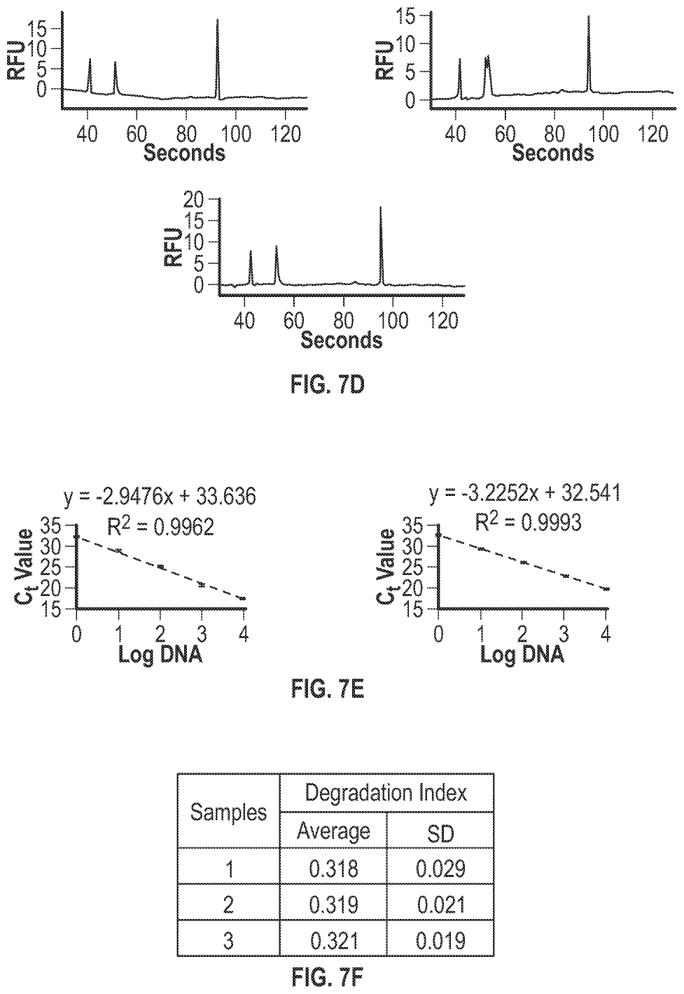

In-Tube Optimization of the Microfluidic Method. The conventional, gold-standard dSP-BSC process is a multi-step workflow requiring several sequential tube transfers, vortexing steps, incubations (both heated and at room temperature), and magnetic manipulations. Given the complexity, in-tube studies were completed prior to microdevice adaptation to isolate each variable for optimal performance at the microfluidic scale. First, samples were prepared with decreased BSC reagent volumes, approximately 1/10^{th} of the manufacturer recommended amount; however, the concentration of silica beads remained consistent, as a reduction in the volume of beads resulted in diminished DNA recovery. Cₜ values originating from samples with decreasing volumes of silica bead solutions from 10 μL to 5 μL and 1 μL show statistical differences overall (one-way ANOVA, α=0.05, p-value=0.0002), with the lowest Cₜ values demonstrated with preparation using 10 μL volumes (32.09±1.76). Likewise, the elution volume was kept consistent to ensure a large enough volume for downstream testing. Regarding remaining BSC reagents, unpaired t-tests comparing Cₜ values originating from samples bisulfite converted with full and microfluidic volumes show no statistical differences for non-methylated and methylated control samples (α=0.05, p-values=0.3803 and 0.1016, respectively), indicating similar recovery FIG. 7C. Likewise, non-methylated control samples, for which all cytosines would ostensibly be converted to uracil, produced statistically similar HRM values (unpaired t-test, α=0.05, p-value=0.248) and were consistent with the known Tₘ for that locus FIG. 7D, indicating comparable conversion efficiency. Dissimilarly, a comparison of the Tₘ values for the methylated controls converted with different conditions showed statistical differences (unpaired t-test, α=0.05, p-value=0.0265); however, the difference between means was only calculated to be ˜0.266° C. (±0.11° C.) FIG. 7C. These results were considered acceptable and further in-tube optimization to decrease dwell temperatures and intervals was completed with microfluidic volumes.

Three incubation parameters were optimized at the microfluidic scale to increase adaptability of the protocol to our microfluidic system and reduce total analytical time, including 1) denaturation, 2) sulphonation and deamination, and 3) desulphonation.

Conventional denaturation parameters necessitated an 8 min incubation at 98° C.; this parameter was reduced first from 8 min to 1 min with no difference in estimated recovery (unpaired t-test, α=0.05, p-value=0.0972). Dwell temperature was subsequently decreased from 98° C. to 96.5° C. and 95° C. for 1 min; likewise, one-way ANOVA results indicated no statistical differences between eluates produced with decreasing dwell temperature overall (α=0.05, p-value=0.0511) (FIG. 7E). For successive studies, samples were converted at 95° C. for 1 min, parameters much more amenable to microfluidic integration. For optimization of the next incubation step, sulphonation and deamination internals were reduced from 60 min to 45, 30, and 15 minutes; interestingly, samples prepared via the conventional protocol (e.g., 60 min incubation) showed higher Cₜ values (34.60±0.98) than those incubated for only 45 min (30.67±1.75), indicating recovery was enhanced by reducing the incubation time (FIG. 7E). This trend was reversed when samples were only incubated for 30 or 15 min, likely as a result of incomplete conversion and corresponding primer mismatch during amplification. In fact, (FIG. 4F) shows the corresponding Tₘ values, providing evidence of incomplete conversion as incubation time decreased lower than a 45 min interval. Also evident from this figure is a statistical difference between Tₘ values undergoing sulphonation and deamination for 60 or 45 min (α=0.05, p-value=0.027), with 45 min incubated samples providing lower Tₘ values, indicating potentially increased conversion efficiency. Notably, samples undergoing sulphonation and deamination for less than 45 minutes showed either reduced recovery (FIG. 4E) or conversion efficiency (FIG. 4F). Thus, to test the optimal conversion time of the final reaction interval, desulphonation, a 45 min sulphonation and deamination was used. For desulphonation, the ideal interval in terms of both recovery and conversion efficiency was determined to be the conventional one of 20 min, producing eluates with the lowest Cₜ (FIG. 4C) and Tₘ values (FIG. 4C). In summary, the BSC parameters have been adapted for microfluidic integration, with decreased reagent volumes (e.g., 1/10^{th} of the standard workflow, not including silica beads), and reduced shortened to 45 min total.

Moving forward, the on-disc workflow incorporated all optimized parameters discussed above; additionally, wash evaporation time was reduced from 20 min to 1 min, as no wash buffer was visibly detected with on-disc evaporation at 55° C. for the shortened interval, reducing the entire workflow by ˜36.61% compared to the gold-standard method.

Microdevice Design. The rotationally-driven μCD was designed for multiplexed analysis of up to four samples in parallel. Each domain includes all the necessary architectural features to support the sequential unit operations associated with the dSP-BSC workflow, wherein all of the architecture situated toward the center of rotation from the magnetic manipulation chamber houses the aqueous reagents, and the chambers closer to the edge of the disc accommodate reaction waste and the final BSC eluate. Initial conversion steps, including denaturation and sulphonation and deamination, are completed in the bisulfite conversion chamber. Following these steps, the partially converted material and aqueous buffer is spun into the magnetic manipulation chamber featuring the dynamic solid phase and a chaotropic solution to promote DNA-silica bead interactions. Note that the concave-shaped magnetic manipulation chamber was designed to retain magnetic beads during waste removal and was previously optimized elsewhere. Both the bisulfite conversion and magnetic manipulation chambers undergo heated incubations that may cause thermal pumping and subsequent fluid loss; thus, each of these chambers feature a hydrophobic membrane composed of polytetrafluoroethylene (PTFE) on the vent and a ‘closed’ loading arm channel. The device makes use of sacrificial valves to enable sequential unit operations, making each device single-use and preventing the potential for contamination and device failure from repeated use. The valving strategy is depicted in the schematic shown in FIG. 5D. This approach makes use of an optically-dense intermediate layer at the center of the disc that is thermally ablated by an external laser to form a pinhole, permitting fluid to flow from layer 2 to layer 4. To subsequently close channels and prevent backflow, the laser is positioned upstream from the opened valve, and laser parameters, including output power, contact time, and height from the surface of the disc, are altered to thermally deform and occlude flow. The precise parameters for both valve opening and channel or ‘valve’ closing are detailed hereinabove with respect to the methods.

Fluidic Control Testing and Characterization. Reliability of the microfluidic BSC method is based upon the reproducibility of the fully-integrated μCD. To assess the architectural features and their ability to complete unit operations during discrete reactions, fluidic dye studies were completed. FIG. 6A shows the progress of one representative dye study as it progresses through each of the BSC steps, including sulphonation and deamination, bead binding, wash steps, desulphonation, and the final DNA elution. Alternating blue and yellow dye solutions were moved throughout each domain of a 4-plex disc through the requisite channels and chambers successfully, indicating fluidic control and reproducibility.

Complete adaptation to the microdevice requires that all incubations be completed on disc. Following the shortening of reaction intervals described above, it stands that the longest on-disc heating interval occurs during the sulphonation and deamination step (54° C. for 45 min), preceded by a brief denaturation in the same chamber (95° C. for 1 min). Upon visual inspection, it appeared some fluid loss was reproducibly occurring during this step (FIG. 6B). A dye study was completed to quantify this loss per a previously described protocol, known as ‘The Crop-Threshold-and-Go’ method of image segmentation and analysis. Here, a calibration curve correlating average pixel area and fluid volume was constructed from digital scans of bisulfite conversion chambers loaded with Allura Red dye at regular intervals, including 1, 5, 7.5, 10, 12.5, and 15 μL (R²=0.9945, y=55.80.2x−621.92) (FIG. 6C). Subsequent scans were taken of the conversion chambers pre-and post-heating, and the corresponding volumes were extrapolated from image analysis and according to their placement along the standard curve. Results indicated that ˜83% of the fluid was retained following this heated incubation step, with pre-and post-heat volumes approximated to be 15.28±0.95 and 12.73±1.28, respectively (FIG. 6D). We can speculate that fluid is being lost to the intermediate layers surrounding the chamber, given all outlets are closed to the external environment. In particular, the chamber vent incorporates a hydrophobic PTFE membrane to prevent fluid loss and the loading port channel is thermally occluded (e.g., ‘closed’) prior to heating (FIG. 6B, inset). Thus, we cannot confirm whether the “leaking” leads to DNA loss from the chamber or simply concentration of the DNA into a reduced aliquot of fluid.

Microdevice Testing with Methylation Standards. To compare the performance of the conventional ‘gold-standard’ method and the optimized on-disc method, non-methylated controls were converted at equivalent concentrations and subsequently assessed for relative DNA recovery and conversion efficiency. Post-BSC, the total amount of DNA in each corresponding amplification reaction totaled 100, 10, and 1 ng (e.g., 4 ng/μL, 400 pg/μL, and 40 pg/μL, respectively); unpaired t-tests of resultant Cₜ values were not statistically different at each concentration (α=0.05, p-values=0.6083, 0.0804, 0.4596, respectively), indicating similar recovery between the gold-standard and on-disc methods (FIG. 7A). Equivalent concentrations of non-methylated standards were also prepared in-tube using the microfluidic volumes and incubation parameters. Relative recovery results indicate similar recovery across conditions for samples prepared with DNA input amounts of 10 and 1 ng total (unpaired t-tests, α=0.05, p-values=0.5368 and 0.3693, respectively); however, the in-tube microfluidic method demonstrated markedly increased recovery compared to the on-disc method at 100 ng total (unpaired t-test, α=0.05, p-value=<0.0001), with average Cₜ values of 28.89±0.51 and 36.23±2.89, respectively. This may indicate the potential of the microfluidic method at concentrations higher than 4 ng/μL with optimal microdevice performance. At this point, the microdevice provides a faster, automated BSC alternative that performs comparably in terms of DNA recovery, and with only ˜ 1/10^{th} of the reagent volumes, theoretically decreasing cost at scale.

As before, relative conversion efficiency was assessed with HRM following the RT-PCR reaction. While no statistical difference was determined at the higher concentration (unpaired t-test, α=0.05, p-value=0.5477), differences were observed with the lower DNA input amounts, including 10 ng (unpaired t-test, α=0.05, p-value=0.0152), and 1 ng (unpaired t-test, α=0.05, p-value=0.0014). However, the differences between Tₘ values were negligible overall; on average, differences between 10 ng eluates ranged 0.55° C.±0.20° C. and 1 ng eluates were only different by 0.32° C. ±0.08° C. (FIG. 7B). Comparing these results to the same concentrations of standards prepared using the in-tube microfluidic method trends reverse with no statistical difference at the lowest concentration (unpaired t-test, α=0.05, p-value =0.0001) and noticeable differences at the higher DNA input amounts of 100 and 10 ng total (unpaired t-tests, α=0.05, p-values=<0.0001 and 0.0001, respectively). At 100 ng total, the in-tube microfluidic method exhibits lower Tₘ values of 0.68° C.±0.14° C. compared to its on-disc counterpart, once again indicating the potential of the microfluidic scheme, if fully optimized to reduce fluid loss. Overall, the standard deviations, or spread, of Tₘ values was the lowest with the automated, on-disc method when comparing all conditions and concentrations, speaking to the reproducibility of this mode. Diving deeper into the variation across BSC preparation conditions and the estimation of conversion efficiency, at the highest DNA input amount (e.g., 100 ng total in the PCR reaction), an additional peak was reproducibly observed with HRM (FIG. 7C). Generally, multiple melt curves suggest nonspecific amplification; however, the NTCs did not indicate contamination and the additional ‘peak’ exhibited low amplitude and appeared broad and unresolved (FIG. 7C). This brings up a shortcoming of HRM, whereby an assumption is made that DNA melting is a 2-stage process resulting in only the detection of amplicons in their double-and single-stranded states. In reality, there may often be an intermediate state wherein the G/C rich portions of the amplicon maintain a double-stranded configuration and A/T rich regions disassociate first. To confirm this phenomenon with the FHL2 amplicons at the highest concentrations and with on-disc BSC eluates, resultant amplicons were separated via microchip electrophoresis. Results indicate the presence of only one amplicon at 133 base pairs (bp), as anticipated, and suggest a multi-stage melt may occur at higher concentrations with this particular target (FIG. 7D). This may also account for the variation observed here between Tₘ values across all sample preparation conditions at 100 ng total.

To evaluate the potential for DNA degradation resulting from conversion-related fragmentation, the Quantifiler Trio DNA Quantification Kit was used. This kit is typically used in forensic DNA analysis workflows to quantify DNA, test for the contribution of male genetic material, and assess the quality of forensic samples that are often subject to environmental influences that lead to nucleic acid degradation. Degradation indices are automatically calculated by the associated software and based upon Cₜ values of diluted standards for large and small autosomal targets. Here, R² values were high (>0.99) (FIG. 7E) and the associated Internal PCR Control (IPC) amplified as expected, indicating the amplification reaction was not affected by any inhibitors and efficiency was as expected. Calculated degradation indices from non-methylated DNA standards converted on-disc via the μCD method are <1, indicating that the DNA is not degraded or inhibited. Additionally, indices are fairly consistent between conversion replicates and indicate consistency with regard to degradation (FIG. 7F). These results are both relevant to forensic use of the workflow and confirm that degradation via the μCD method should not interfere with interpretation at this concentration (˜1.25 ng/μL).

Comparison to an Enzymatic Method for Cytosine Deamination. In response to the aforementioned issues associated with gold-standard sodium bisulfite conversion, namely DNA fragmentation and loss, alternative methods for the conversion of cytosines for epigenetic analysis have been developed commercially. One such commercialized method forgoes chemical conversion and relies upon an apolipoprotein B mRNA-editing enzyme, catalytic peptide (APOBEC) for the deamination of cytosine to uracil, leaving modified cytosines (e.g., 5-methylcytosines and 5-hydroxymethylcytosines) intact via enzymatic modification by a ten-eleven translocation 2 (TET2) enzyme and Oxidation Enhancer. To compare the results from μCD dSP-BSC with this alternative method for conversion, non-methylated control DNA was enzymatically converted at equivalent amounts of input DNA, as before. In examining the results from duplicate enzymatic conversion reactions in terms of relative DNA recovery and conversion efficiency, respectively. Generally, relative recovery results were inconsistent in comparison with the μCD method, indicating that the microdevice method showed greater DNA recovery at total DNA input amounts of 100 ng (μCD mean Cₜ values 5.42±2.13 lower) and lower recovery at 10 ng total (μCD mean Cₜ values 3.37 ±1.26 higher. While recovery at 1 ng total, perhaps the most forensically-relevant range, was found to show no statistical differences between average Cₜ values (unpaired t-test, α=0.05, p-value=0.4174). However, this is likely the result of stochastic differences across all samples processed within this concentration range; a direct comparison of units reveals a lower mean Cₜ difference of 1.28±0.06 for samples prepared via the microdevice, indicating overall higher recovery. When comparing Tₘ values associated with the enzymatic approach, temperatures are statistically different across concentrations (one-way ANOVA, α=0.05, p-value=<0.0001), potentially indicating that DNA input amount influences conversion efficiency. However, in estimations of conversion efficiency via HRM, the enzymatic method outperformed the microfluidic approach by a mean temperature difference of 1.02±0.11° C.; these differences were also found to be statistically significant (unpaired t-test, α=0.05, p-value=<0.0001). In total, preliminary results comparing the μCD and enzymatic approaches indicate that performance is likely dependent upon DNA input amount with regard to recovery and slightly improved in terms of conversion efficiency with the enzymatic method. Of course, the data set and analytical range tested here is relatively small and further testing is required for a true comparison. With regard to manual intervention and time at the bench, the enzymatic approach required DNA pre-processing (e.g., shearing), 11 more reagents and associated manual handling steps/tube transfers, and 6 additional hours of processing time compared to the μCD method.

The chemical modification of cytosine residues to uracil via sodium bisulfite conversion has remained largely steadfast since its conception several decades ago and is widely accepted to be associated with DNA degradation and loss. For the preponderance of epigenetic applications, this loss may be compensated for by using samples known to contain higher concentrations of nucleic acids and/or by incorporating upstream enrichment techniques to increase DNA concentration from a large volume of sample. Unfortunately, forensic casework samples are known to have limited DNA contributions that are often fragmented for a number of reasons, including limited sample deposits, environmental exposure, or sample partitioning for individualizing identification efforts, to name a few. Thus, applications in forensic epigenetics, wherein DNA is subject to an additional, deleterious sample preparation process post-extraction, may result in complete loss of the sample and are therefore not ideal for integration with the forensic workflow in their current form. Additionally, the adage of another ‘open-tube’ process with several labor-intensive pipetting steps increases time at the bench, the risk for contamination, and opportunities for errors by the analyst.

The instant disclosure provides a microfluidic solution for forensic epigenetic sample preparation that decreases contamination risks and the potential for interoperability issues that are often associated with manual handling. By leveraging decreased, microfluidic volumes, the described method enables reduced incubation times by ˜36%, and preliminary results indicate increased recovery compared to a gold-standard method. The μCD itself incorporates centrifugal force and sacrificial, laser-based valving for fluidic control and the performance of discrete unit operations, permitting automation, reproducibility, and a small overall footprint for preparation of up to four samples in parallel. The fully-integrated device does exhibit some fluid loss through uptake to the surrounding material during the longest incubation step (e.g., sulphonation and deamination) that may be associated with loss of sensitivity compared to an in-tube microfluidic approach; yet, when comparing controls converted with both gold-standard and on-disc approaches at multiple concentrations, there are no statistical differences in recovery and only negligible differences in conversion efficiency. Likewise, samples prepared via the μCD show no evidence of DNA degradation or inhibition from residual reagents (e.g., ethanol) in the converted eluate, as indicated by a commercial kit intended for forensic characterization of these particular factors. Finally, in a limited comparison of the μCD method and an alternative, enzymatic approach for cytosine conversion, the results were largely stochastic, but indicate that DNA input concentration may be a key factor of performance. Additionally, the enzymatic method necessitated shearing the DNA up front for successful conversion and required a 300× increase in time at the bench, several manual handling steps, and 11 more reagents when compared with the μCD approach. In summary, this work demonstrates progress toward a microfluidic sodium bisulfite conversion method that is more amenable to integration with the forensic DNA workflow but will benefit from further validation and characterization in the future.

### Example 2

### Characterization of the dSP-BSC Microdevice

Prior to integrating the method onto the disc (as shown in FIG. 5 of Example 1), it was essential to ensure the conversion chemistry would perform at the microfluidic scale. To test this, 0% and 100% methylated standard samples were prepared in parallel according to manufacturer's recommendations and at the microfluidic scale, using approximately 1/10^{th} of the recommended reagent volumes. Resultant converted eluates were amplified by real-time polymerase chain reaction (PCR) and an intercalating fluorescent dye (i.e., Syto 9) using the QuantStudio 5 System at a final PCR concentration of 5 ng/μL.

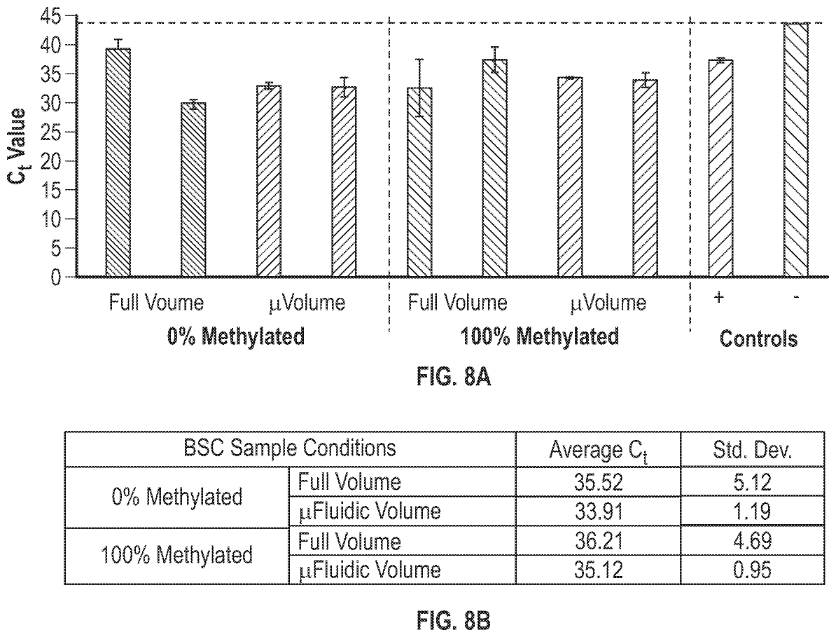

Resultant cycle threshold (Cₜ) values suggest that samples prepared using the microfluidic sample volumes (μvolume) were higher in DNA concentration, indicating higher recovery post conversion (FIG. 8A). Upon closer examination of replicate converted samples, it appears that standard deviations are considerably smaller with μvolume when compared to full volume reactions; likely, this is a result of increased stochastic effects with larger volume samples.

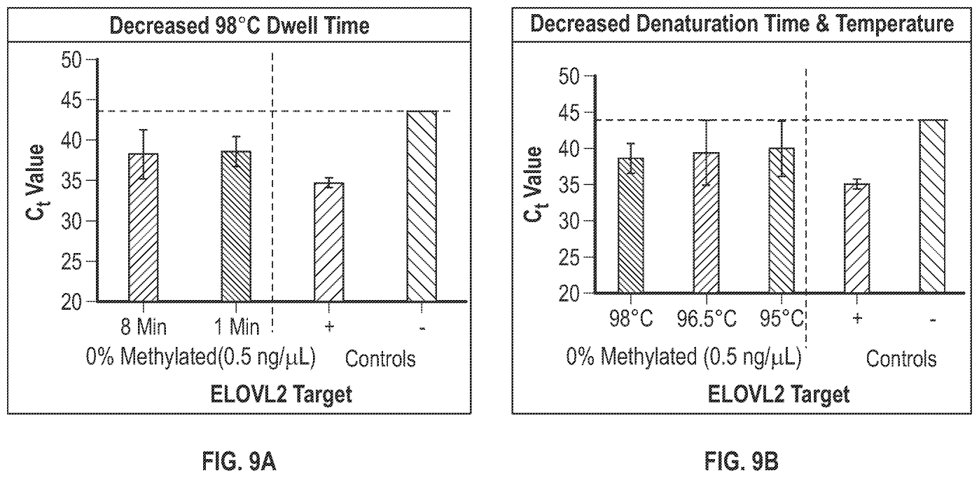

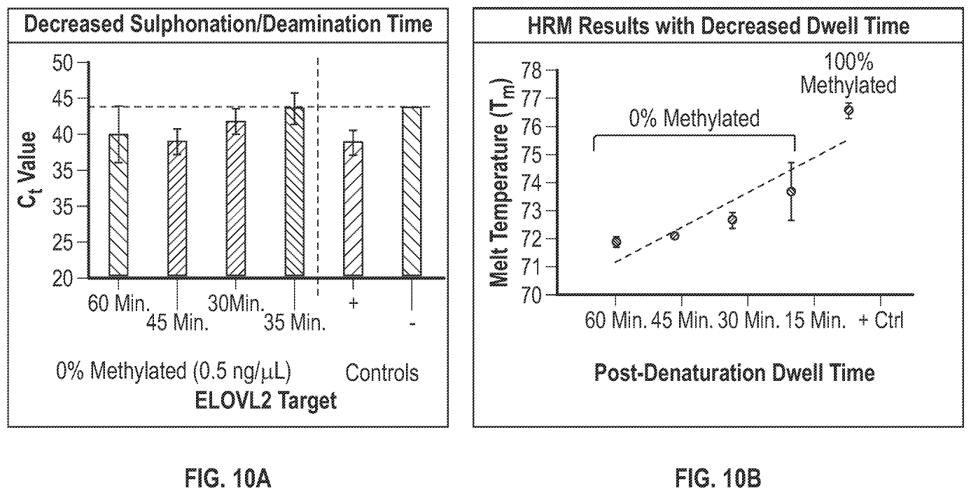

Methylated standard samples at 0% status were selected for ongoing studies as they would require conversion at all cytosine positions, giving us more perspective into how alterations of the chemistry would affect recovery and efficiency with DNA that would be most affected by the reaction. With confirmation that the sample preparation assay performs at the microfluidic scale, it was necessary to make the process more amenable to microfluidic integration and DNA recovery. Namely, this required decreasing the temperature and times required for incubation. Referring back at FIG. 9, the first step of conversion, Sulphonation and Hydrolytic Deamination, requires incubations at 98° C. and 54° C. for 8 mins and 60 mins, respectively. As a result of the decreased surface-area-to-volume ratio provided by the microfluidic platform, as compared to the in-tube method, the present inventor also believed it possible to also shorten some of the associated incubation times and the initial 98° C. incubation temperature, thus shortening total assay time and limiting DNA loss. Testing this began with the in-tube proof-of-principle reduction of initial denaturation time from 8 mins down to 1 min (FIG. 8A). Further, the temperature of the initial incubation was decreased from 98° C. to 96.5° C. and 95° C. (FIG. 8B). Results suggest there is no statistical difference produced between resultant Cₜ values from eluates prepared with altered denaturation conditions. Note that standard deviations are characteristically high of the method and in the stochastic range of 0.5 ng/μL, a concentration selected to mimic forensic casework level concentrations of nucleic acid.

To further shorten total dSP-BSC assay time, the second 54° C. incubation was shortened from 60 min to 45, 30, and 15 min and samples were amplified and melting using high resolution melting (HRM) analysis at a PCR concentration of 0.5 ng/μL. The present inventor hypothesized that shortening this step would be potentially problematic, as residues must be incubated long enough to form their second intermediary structure, but not so long as to become subject to degradation. Results for real-time PCR are shown in FIG. 9A and suggest some diminished recovery with the full 60 min incubation and increased performance with 45 min incubation. However, this trend quickly reversed for samples incubated for only 30 and 15 min. Recovery aside, it was important to understand how shortening this step would effect cytosine conversion; if we look at the graph shown in FIG. 9B as a spectrum, with the 100% methylated control melting around 77° C. and the converted 0% samples residing around 72° C., we see a trend for samples to increase in melt temperature as their dwell time is decreased. This trend indicates a higher GC content, or lower conversion rate, as the dwell times are decreased past 45 minutes.

As a final proof-of-concept study, the first stage of the dSP-BSC process was performed using the μCD and a mechatronic system equipped with a heating element (e.g., a dual-clamp Peltier) using the forementioned time and temperature specifications (e.g., 95° C. for 1 min and 54° C. for 45 min). Post-initial conversion steps including silica bead washing, desulphonation, and sample elution were performed in-tube to ensure results could be appropriately compared with all in-tube data. Real-time PCR results suggest μCD processing is robust at two low concentrations (e.g., 2.5 ng/μL and 0.5 ng/μL) for initial conversion.

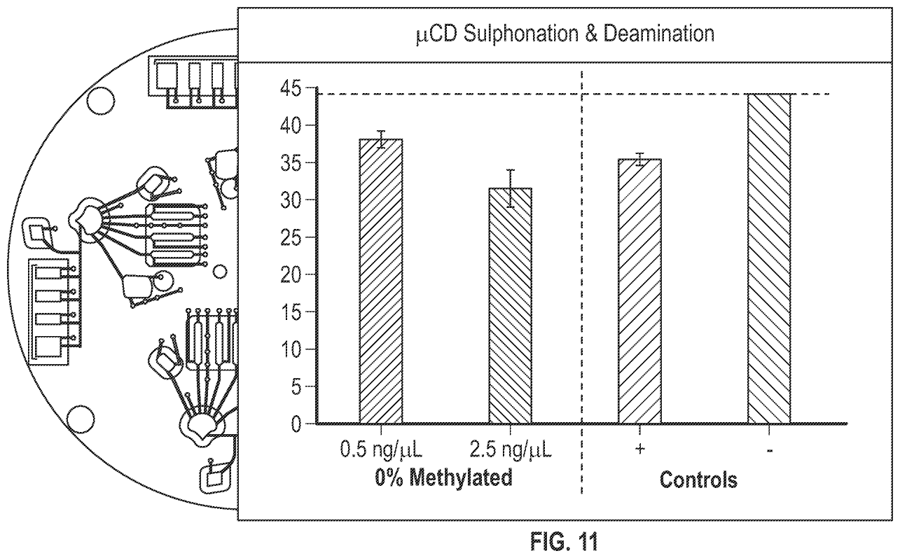

With completed dye studies to suggest the proposed architecture (FIG. 11) will facilitate sequential unit operations (data not shown), continued validation of the dSP-BSC method will include completing the entire chemical workflow on disc to demonstrate its robusticity. The analytical bounds of the technique will be investigated with the completion of several studies involving the method's reproducibility, sensitivity, and performance compared to the in-tube method. Further, eluates will be analyzed with analytical techniques beyond real-time PCR and HRM, including capillary electrophoresis (CE) and pyrosequencing; the optimization of those strategies is currently underway.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Aspect 1 provides a centrifugal microfluidic device to perform dynamic solid phase sodium bisulfate conversion, the device comprising:
a reaction assembly, comprising:
   a plurality of individual chambers comprising:
      a bisulfate conversion chamber;
      an elution chamber;
      a magnetic manipulation chamber;
      a waste chamber; and
      a buffer chamber; and
at least one valve configured to selectively establish or prevent fluid communication along a channel between at least two respective individual chambers amongst the plurality of individual chambers;
wherein the reaction assembly is configured to establish fluidic transport in response to rotation about an axis intersecting a central region of the reaction assembly and perpendicular to a plane on which the reaction assembly is disposed.

Aspect 2 provides the centrifugal microfluidic device of Aspect 1, wherein the reaction assembly is a first reaction assembly and the device further comprises a second reaction assembly disposed coplanar with the first reaction assembly.

Aspect 3 provides the centrifugal microfluidic device of Aspect 2, further comprising:
a third reaction assembly disposed coplanar with the first reaction assembly and the second reaction assembly; and
a fourth reaction assembly disposed coplanar with the first reaction assembly, the second reaction assembly, and the third reaction assembly.

Aspect 4 provides the centrifugal microfluidic device of any of Aspects 2 or 3, wherein at least two of the first reaction assembly, second reaction assembly, third reaction assembly, and fourth reaction assembly are identically constructed.

Aspect 5 provides the centrifugal microfluidic device of any of Aspects 1-4, wherein
the magnetic manipulation chamber is located more distally to the central region relative to the bisulfate elution chamber and the waste chamber;
the bisulfate conversion chamber is located more distally to the central region relative to the magnetic manipulation chamber; and
the buffer chamber is located more distally to the central region relative to the bisulfate conversion chamber, the axis defining a center-of-rotation.

Aspect 6 provides the centrifugal microfluidic device of any of Aspects 1-5, wherein the buffer chamber is a first buffer chamber and the device further comprises at least a second buffer chamber.

Aspect 7 provides the centrifugal microfluidic device of any of Aspects 1-6, wherein the buffer chamber is a wash buffer chamber, an eluate buffer chamber, or a desulphonation buffer chamber.

Aspect 8 provides the centrifugal microfluidic device of any of Aspects 1-6, wherein the at least one valve is configured to selectively establish or prevent fluid communication in response laser irradiation.

Aspect 9 provides the centrifugal microfluidic device of any of Aspects 1-8, wherein the device comprises a plurality of stacked layers.

Aspect 10 provides the centrifugal microfluidic device of Aspect 9, wherein the bisulfate conversion chamber, the bisulfate elution chamber, the magnetic manipulation chamber, the waste chamber, and the buffer chamber are defined by laser etching.

Aspect 11 provides the centrifugal microfluidic device of any of Aspects 1-10, further comprising a processor communicatively coupled with a mechanical actuator, process configured to control the mechanical actuator to establish the fluidic transport.

Aspect 12 provides an in situ method for performing dynamic solid phase sodium bisulfate conversion, the method comprising:
feeding a nucleic acid sample into a device, wherein device comprises: a reaction assembly, comprising:
a plurality of individual chambers comprising:
   a bisulfate conversion chamber;
      an elution chamber;
      a magnetic manipulation chamber;
      a waste chamber; and
      a buffer chamber; and
      at least one valve configured to selectively establish or prevent fluid communication along a channel between at least two respective individual chambers amongst the plurality of individual chambers;
         wherein the reaction assembly is configured to establish fluidic transport in response to rotation about an axis intersecting a central region of the reaction assembly and perpendicular to a plane on which the reaction assembly is disposed
reacting the nucleic acid sample with sodium sulfate to form a partially sulphonated nucleic acid;
spinning the device to move the partially sulphonated nucleic acid to the magnetic manipulation chamber to contact the partially sulphonated nucleic acid with a magnetic bead;
deaminating and desulphonating the partially sulphonated nucleic acid in the magnetic manipulation chamber;
contacting the deaminated and desulphonated nucleic acid with an elution buffer to form an eluted product; and
spinning the device to move the eluted product to the elution chamber.

Aspect 13 provides the method of Aspect 12, wherein the reaction assembly is a first reaction assembly and the device further comprises a second reaction assembly disposed coplanar with the first reaction assembly.

Aspect 14 provides the method of Aspect 13, further comprising:
a third reaction assembly disposed coplanar with the first reaction assembly and the second reaction assembly; and
a fourth reaction assembly disposed coplanar with the first reaction assembly, the second reaction assembly, and the third reaction assembly.

Aspect 15 provides the method of any of Aspects 13 or 14 wherein at least two of the first reaction assembly, second reaction assembly, third reaction assembly, and fourth reaction assembly are identically constructed.

Aspect 16 provides the method of any of Aspects 12-15, wherein the magnetic manipulation chamber is located more distally to the central region relative to the bisulfate elution chamber and the waste chamber;
the bisulfate conversion chamber is located more distally to the central region relative to the magnetic manipulation chamber; and
the buffer chamber is located more distally to the central region relative to the bisulfate conversion chamber, the axis defining a center-of-rotation.

Aspect 17 provides the method of any of Aspects 12-16, wherein the buffer chamber is a first buffer chamber and the device further comprises at least a second buffer chamber.

Aspect 18 provides the method of any of Aspects 12-17, wherein the buffer chamber is a wash buffer chamber, an eluate buffer chamber, or a desulphonation buffer chamber.

Aspect 19 provides the method of any of Aspects 12-18, wherein the at least one valve is configured to selectively establish or prevent fluid communication in response laser irradiation.

Aspect 20 provides the method of any of Aspects 12-19, wherein the device comprises a plurality of stacked layers.

Aspect 21 provides the method of Aspect 20, wherein the bisulfate conversion chamber, the bisulfate elution chamber, the magnetic manipulation chamber, the waste chamber, and the buffer chamber are defined by laser etching.

Aspect 22 provides the method of any of Aspects 12-21, further comprising amplifying the eluted nucleic acid.

Aspect 23 provides the method of any of Aspects 12-22, wherein the nucleic acid is DNA.

Aspect 24 provides the method of any of Aspects 12-23, wherein the device is under the control of a processor communicatively coupled with a mechanical actuator, process configured to control the mechanical actuator to establish the fluidic transport.

Aspect 25 provides a method for performing any combination of one or more of the following: a) rotationally-driven microfluidic technique for DNA methylation sample preparation by dynamic solid phase sodium bisulfite conversion; b) optimization of a rationally-driven microfluidic technique for forensic DNA methylation sample preparation (or other DNS applications besides forensic DNA); c) microfluidic technique for DNA methylation sample preparation; d) Sodium Bisulfite Conversion (BSC) technique to enable incorporation of forensic DNA phenotyping (FDP), and ultimately reference-free externally visible characteristics (EVC) analysis, into forensic casework (or other applications besides forensic); e) centrifugal microfluidic platform technique for dynamic Solid Phase sodium Bisulfite Conversion (dSP-BSC) that enables rapid, efficient conversion of smaller, forensically-relevant DNA input masses in an automated, closed system (or other applications besides forensically-relevant DNA); f) fully integrated, automated, and enclosed system technique that minimizes both variability and contamination risk through automation and integration to supplant the manual, open-tube steps otherwise required in traditional Sodium Bisulfite Conversion (BSC) methods; or g) rotationally-driven technique for silica-facilitated sodium bisulfite conversion for downstream nucleic acid analysis, as described herein.

Aspect 26 provides the method according to Aspect 25, including each and every novel feature or combination of features disclosed herein.

Aspect 27 provides a system for providing any combination of one or more of the following: a) rotationally-driven microfluidic system for DNA methylation sample preparation by dynamic solid phase sodium bisulfite conversion; b) optimization of a rationally-driven microfluidic system for forensic DNA methylation sample preparation (or other DNS applications besides forensic DNA); c) microfluidic system for DNA methylation sample preparation; d) Sodium Bisulfite Conversion (BSC) technique to enable incorporation of forensic DNA phenotyping (FDP), and ultimately reference-free externally visible characteristics (EVC) analysis, into forensic casework (or other applications besides forensic); e) centrifugal microfluidic platform for dynamic Solid Phase sodium Bisulfite Conversion (dSP-BSC) that enables rapid, efficient conversion of smaller, forensically-relevant DNA input masses in an automated, closed system (or other applications besides forensically-relevant DNA); f) fully integrated, automated, and enclosed system that minimizes both variability and contamination risk through automation and integration to supplant the manual, open-tube steps otherwise required in traditional Sodium Bisulfite Conversion (BSC) methods; or g) rotationally-driven device for silica-facilitated sodium bisulfite conversion for downstream nucleic acid analysis, as described herein.

Aspect 28 provides the system according to Aspect 27, including each and every novel feature or combination of features disclosed herein.

Aspect 29 provides a computer-readable storage medium having computer-executable instructions stored thereon which, when executed by one or more processors, cause one or more computers to perform functions for performing any combination of one or more of the following: a) rotationally-driven microfluidic technique for DNA methylation sample preparation by dynamic solid phase sodium bisulfite conversion; b) optimization of a rationally-driven microfluidic technique for forensic DNA methylation sample preparation (or other DNS applications besides forensic DNA); c) microfluidic technique for DNA methylation sample preparation; d) Sodium Bisulfite Conversion (BSC) technique to enable incorporation of forensic DNA phenotyping (FDP), and ultimately reference-free externally visible characteristics (EVC) analysis, into forensic casework (or other applications besides forensic); e) centrifugal microfluidic platform technique for dynamic Solid Phase sodium Bisulfite Conversion (dSP-BSC) that enables rapid, efficient conversion of smaller, forensically-relevant DNA input masses in an automated, closed system (or other applications besides forensically-relevant DNA); f) fully integrated, automated, and enclosed system technique that minimizes both variability and contamination risk through automation and integration to supplant the manual, open-tube steps otherwise required in traditional Sodium Bisulfite Conversion (BSC) methods; or g) rotationally-driven technique for silica-facilitated sodium bisulfite conversion for downstream nucleic acid analysis, as described herein.

Aspect 30 provides the computer-readable storage medium of Aspect 29, including each and every novel feature or combination of features disclosed herein.

Aspect 31 provides an article of manufacture provided by any combination of one or more of the following: a) rotationally-driven microfluidic method or system for DNA methylation sample preparation by dynamic solid phase sodium bisulfite conversion; b) optimization of a rationally-driven microfluidic method or system for forensic DNA methylation sample preparation (or other DNS applications besides forensic DNA); c) microfluidic method or system for DNA methylation sample preparation; d) Sodium Bisulfite Conversion (BSC) method or system to enable incorporation of forensic DNA phenotyping (FDP), and ultimately reference-free externally visible characteristics (EVC) analysis, into forensic casework (or other applications besides forensic); e) centrifugal microfluidic platform method or system for dynamic Solid Phase sodium Bisulfite Conversion (dSP-BSC) that enables rapid, efficient conversion of smaller, forensically-relevant DNA input masses in an automated, closed system (or other applications besides forensically-relevant DNA); f) fully integrated, automated, and enclosed method or system that minimizes both variability and contamination risk through automation and integration to supplant the manual, open-tube steps otherwise required in traditional Sodium Bisulfite Conversion (BSC) methods; or g) rotationally-driven method or system for silica-facilitated sodium bisulfite conversion for downstream nucleic acid analysis, as described herein.

Aspect 32 provides the article of manufacture according to Aspect 31, including each and every novel feature or combination of features disclosed herein.

1. A centrifugal microfluidic device to perform dynamic solid phase sodium bisulfite conversion, the device comprising:
a reaction assembly, comprising:
a plurality of individual chambers, channels and connections comprising:
a bisulfite conversion chamber;
an elution chamber;
a magnetic manipulation chamber;
a waste chamber; and
a buffer chamber; and
at least one valve configured to selectively establish or prevent fluid communication along single or multiple channels between at least two respective individual chambers amongst the plurality of individual chambers;
wherein the reaction assembly is configured to establish fluidic transport in response to rotation about an axis intersecting a central region of the reaction assembly and perpendicular to a plane on which the reaction assembly is disposed.

2. The centrifugal microfluidic device of claim 1, wherein the reaction assembly is a first reaction assembly and the device further comprises a second reaction assembly disposed coplanar with the first reaction assembly.

3. The centrifugal microfluidic device of claim 2, further comprising:
a third reaction assembly disposed coplanar with the first reaction assembly and the second reaction assembly; and
a fourth reaction assembly disposed coplanar with the first reaction assembly, the second reaction assembly, and the third reaction assembly.

4. The centrifugal microfluidic device of claim 2, wherein at least two of the first reaction assembly, second reaction assembly, third reaction assembly, and fourth reaction assembly are identically constructed.

5. The centrifugal microfluidic device of claim 1, wherein
the magnetic manipulation chamber is located more distally to the central region relative to the bisulfite elution chamber and the waste chamber;
the bisulfite conversion chamber is located more distally to the central region relative to the magnetic manipulation chamber; and
the buffer chamber is located more radially to the central region relative to the bisulfite conversion chamber, the axis defining a center-of-rotation.

6. The centrifugal microfluidic device of claim 1, wherein the buffer chamber is a first buffer chamber and the device further comprises at least a second buffer chamber.

7. The centrifugal microfluidic device of claim 1, wherein the buffer chamber is a wash buffer chamber, an eluate buffer chamber, or a desulphonation buffer chamber.

8. The centrifugal microfluidic device of claim 1, wherein the at least one valve is configured to selectively establish or prevent fluid communication in response laser irradiation.

9. The centrifugal microfluidic device of claim 1, wherein the device comprises a plurality of stacked layers.

10. The centrifugal microfluidic device of claim 9, wherein the bisulfite conversion chamber, the bisulfite elution chamber, the magnetic manipulation chamber, the waste chamber, and the buffer chamber are defined by laser etching.

11. The centrifugal microfluidic device of claim 1, further comprising a processor communicatively coupled with a mechanical actuator, process configured to control the mechanical actuator to establish the fluidic transport.

12. An in situ method for performing dynamic solid phase sodium bisulfite conversion, the method comprising:
feeding a nucleic acid sample into a device, wherein device comprises:
a reaction assembly, comprising:
a plurality of individual chambers comprising:
bisulfite conversion chamber;
an elution chamber;
a magnetic manipulation chamber;
a waste chamber; and
a buffer chamber; and
at least one valve configured to selectively establish or prevent fluid communication along a channel between at least two respective individual chambers amongst the plurality of individual chambers;
wherein the reaction assembly is configured to establish fluidic transport in response to rotation about an axis intersecting a central region of the reaction assembly and perpendicular to a plane on which the reaction assembly is disposed
reacting the nucleic acid sample with sodium bisulfite to form a partially sulphonated nucleic acid;
spinning the device to move the partially sulphonated nucleic acid to the magnetic manipulation chamber to contact the partially sulphonated nucleic acid with a magnetic bead;
deaminating and desulphonating the partially sulphonated nucleic acid in the magnetic manipulation chamber;
contacting the deaminated and desulphonated nucleic acid with an elution buffer to form an eluted product; and
spinning the device to move the eluted product to the elution chamber.

13. The method of claim 12, wherein the reaction assembly is a first reaction assembly and the device further comprises a second reaction assembly disposed coplanar with the first reaction assembly.

14. The method of claim 13, further comprising:
a third reaction assembly disposed coplanar with the first reaction assembly and the second reaction assembly; and
a fourth reaction assembly disposed coplanar with the first reaction assembly, the second reaction assembly, and the third reaction assembly.

15. (canceled)

16. The method of any of claim 12, wherein the magnetic manipulation chamber is located more distally to the central region relative to the bisulfite elution chamber and the waste chamber;
the conversion chamber is located more distally to the central region relative to the magnetic manipulation chamber; and
the buffer chamber is located more distally to the central region relative to the bisulfite conversion chamber, the axis defining a center-of-rotation.

17. The method of any of claim 12, wherein the buffer chamber is a first buffer chamber and the device further comprises at least a second buffer chamber.

18. The method of any of claim 12, wherein the buffer chamber is a wash buffer chamber, an eluate buffer chamber, or a desulphonation buffer chamber.

19. The method of any of claim 12, wherein the at least one valve is configured to selectively establish or prevent fluid communication in response laser irradiation.

20. The method of any of claim 12, wherein the device comprises a plurality of stacked layers.

21. (canceled)

22. The method of claim 12, further comprising amplifying the eluted nucleic acid.

23. (canceled)

24. The method of claim 12, wherein the device is under the control of a processor communicatively coupled with a mechanical actuator, process configured to control the mechanical actuator to establish the fluidic transport.
